(19) United States
(12) Patent Application Publication
(10) Pub. No.: US 2002/0155495 A1
(43) Pub. Date: Oct. 24, 2002

(54) METHOD FOR PRODUCING ARRAYS AND DEVICES RELATING THERETO

(76) Inventor: LARRY S. MILLSTEIN, WASHINGTON, DC (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/529,588

(22) PCT Filed: Oct. 16, 1998
(86) PCT No.: PCT/US98/21860

(51) Int. Cl. G01N 33/53; C03B 37/10

(52) U.S. Cl. 435/7.1; 65/427

(57) ABSTRACT

An invention that relates to arrays, to methods and devices for producing arrays and to methods and devices for using arrays is described. In a particular aspect the invention relates to methods in which array members are aligned in a bundle and the bundle then is sectioned across the alignment to produce replicate arrays. In a further particular aspect the invention relates to arrays of analyte binding reagents. In another particular aspect the invention relates to micro-arrays.

[This application is a continuation in part of U.S. Provisional Application No. 60/062,203 filed on Oct. 16, 1997 the entirety of which, by reference, is herein incorporated.]

Arrays are important in many technologies, and methods to make arrays precisely, efficiently and economically are of widespread importance. Recently, the value of arrays with small dimensions has been recognized and interest is high in finding methods to produce a wide variety of small scale arrays commercially.

Chemical, biochemical, and/or biological assays represent a set of applications for arrays of increasing importance. The use of arrays to carry out such assays illustrates many aspects of arrays, their production and use. Discrete assay devices for a wide variety of physical, chemical, biochemical and biological attributes have become commonplace, both in industrial and consumer applications. Among familiar devices of this type are medical test devices such as “dip sticks” that measure chorionic gonadotropin in over-the-counter pregnancy test kits, and autoanalysers that carry out clinical diagnostic chemistry testing. The use of such devices is expanding rapidly as it becomes possible to monitor an increasing number of properties and substances by highly reliable and accurate tests.

Most devices currently in use are directed to a single discrete test, such as a single assay for a particular compound. Even where a large number of assays is performed on a large number of samples, current methods typically proceed by dividing each sample into separate portions for each test and performing the tests separately. This is true even in some highly sophisticated applications. For instance, blood bank autoanalysers generally split each sample into small aliquots that are analyzed separately to determine each measured property. The same often is true of testing urine for drugs, for instance. This “divide and conquer” approach can be efficient and cost effective; but, it is not necessarily the best way to carry out very large numbers of tests on a large number of samples. The divide and conquer strategy diminishes the sample available to each assay in direct proportion to the number of tests that are performed. Hence, it is disadvantageous for tests that require relatively large sample volumes, such as tests for HIV viral load. Furthermore, the divide and conquer strategy requires separate analysis channels for each test. Complexity thus increases directly in proportion to the number of tests performed. In sum, the divide and conquer approach disadvantageously limits the test-effective sample amount and incurs additive sample manipulation, fabrication and apparatus costs that become increasingly onerous as the number of tests and the number of samples increases.

Disadvantages of divide and conquer strategies are overcome by arrays. For instance, several companies have demonstrated devices for DNA-based diagnosis that have a thousand or more different sequence-specific probes on a single assay surface where each one can be individually addressed. Clearly, it would be difficult and impractical to divide each sample into thousands of aliquots to test individually against all these probes, as required by divide and conquer strategies. Instead, all of these devices separate the sequence specific probes into discrete locations in a defined pattern on a surface and expose all probes to the sample at the same time. Results are determined by detecting where the sample hybridizes to the array. All the probes access the entire sample, avoiding the dilution-by-aliquoting effect of divide and conquer strategies. And, the sample is hybridized to all the probes in a single reaction, greatly simplifying the process and reducing its cost. All-at-once approaches using arrays clearly are more effective than divide and conquer strategies for carrying out a large number of assays on only a limited sample. The approach has even more impressive benefits for carrying out a large number of assays on a large number of samples. Widespread availability of arrays thus would be of great benefit in this regard.

Unfortunately it has been possible to make arrays for such applications only by two relatively inefficient and difficult methods: spotting and positional solid phase synthesis. A variety of devices have been used to make arrays by spotting materials onto a surface, including contact spotters and ink jet-like spotters. A contact spotter has been designed and employed by Brown and colleagues at Stanford University to make DNA probe arrays on various surfaces, typically for profiling expression of many genes at once. (See, for instance, the Brown web page at http://cmgm.stanford.edu/pbrown.). This spotter, and other spotter designs, also have been used by many genomics and expression profiling companies, including but not limited to Incyte, Incyte/Synteni, Hyseq and Millenium. (See the web pages and literature of the companies.) For example, scientists at Hyseq reported spotting 8,192 oligonucleotide probes for sequencing-by-hybridization onto a flat surface using a contact spotter of this type. (See Drmanac et al., Nature Biotechnology 16: 54-58 (1998) which is herein incorporated by reference in its entirety.) In an example of arraying using ink jet-like devices, oligonucleotide probes for a hybridization assay were dispensed by a micro-ink jet spotter directly onto the surface of a CCD. (See Eggers et al., Bio Techniques 17: 516-524 (1994) which is herein incorporated by reference in its entirety). Finally, oligonucleotide probes have been spotted onto flow-through chips using both contact spotting and ink jet methods, as described by Beattie and colleagues and developed by Gene Logic. (See, for instance, Beattie et al., WO 95/11755 which is herein incorporated by reference in its entirety.)

The spotting approach requires machinery capable of flawlessly depositing precise volumes of material at precise locations on a substrate, repeatedly. To make 10,000 array replicates of a 10,000 member array using spotting methods requires 100,000,000 spots and, therefore, at least 100,000,000 spotting operations. Accuracy and precision are very difficult to maintain over such a large number of operations, needless to say, and this can limit the use of spotting approaches, particularly for large scale production of complex arrays. In addition, spot size probably cannot be reduced below about 100 microns in diameter in practical spotting devices. If so, spotted arrays will be limited to densities of less than 10,000 assays per square centimeter and will not be suitable for many applications.

A second approach that has been used to make assay arrays involves solid phase synthesis on a surface controlled by photo lithographic techniques. Using this type of approach, the leaders in this field, Affymax and Affymetrix, have used light-addressable peptide and oligonucleotide solid phase synthesis chemistries to build up checkerboard-like arrays of short peptides or oligonucleotides. Affymax scientists initially reported synthesis of an array of 1,024 peptides and since then they have reported much larger arrays. (See, for instance, Fodor et al., Science 251: 767-773 (1991), Pirrung et al., WO 90/15070 and Pirrung et al., U.S. Pat. No. 5,143,854 issued Sep. 1, 1992, which are herein incorporated by reference in their entirety.) Affymetrix scientists have reported a variety of oligonucleotide arrays. (For early results see Pease et al., PNAS 91 5022-5026 (1994) which is incorporated by reference herein in its entirety.) One set of four arrays produced by Affymetrix included 20 pairs of 25-mer oligonucleotide probes for all 6,200 genes predicted by analysis of a complete yeast genomic sequence. (See Wodicka et al., Nature Biotechnology 15: 1359-1367 (1997) which is incorporated by reference herein in its entirety). The arrays included over 250,000 oligonucleotides and were used, initially, to profile expression of all yeast genes all-at-once in single hybridization experiments. The same arrays also have been used to analyze genetic selections in yeast. (See Cho et al., PNAS 95: 3752-3757 (1998) which is incorporated by reference herein in its entirety). Smaller scale arrays of this type have been made for profiling expression of genes of other organisms, such as E. coli and humans. (See, for instance, Saizieu et al., Nature Biotechnology 16: 4548 (1998) and Lockhart et al., Nature Biotechnology 14: 1675-1996 (1996), which are herein incorporated by reference in their entirety.) Arrays like these are used intensively in drug discovery programs to identify disease and therapy associated changes in gene expression and to identify promising targets for drug discovery and development. Affymetrix scientists also have reported oligonucleotide arrays useful for SNP discovery and screening. These include arrays for discovering human SNPs and arrays for sequencing human mitochondrial DNA. (See, for instance, Wang et al., Science 280: 1077-1082 (1998) and Chee et al., Science 274: 610-614 (1996).) The array for mitochondrial sequencing contained 135,000 oligonucleotide probes able to interrogate and in most cases determine the complete sequence of human mitochondrial DNA in a sample, in a single experiment. Arrays of this type are particularly useful for SNP mapping and for pharmacogenomics studies. Both types of arrays demonstrate the power and potential of arrays for all-at-once determinations on a large scale.

Another array-making approach involving combinatorial synthesis of oligonucleotides on a surface was developed by Southern. (See, for instance, Milner et al., Nature Biotechnology 15: 537-541 (1997) which is incorporated by reference herein in its entirety). In this approach, an annular mechanism delivers reagents to selected areas of a surface in a series of addition reactions. The particular overlap of reagent exposures at various locations on the surface defines the array members.

The synthesis approach has disadvantages, however, despite the aforementioned success. First, assay diversity in this approach depends on combinatorial build-up chemistries. Because of this it is limited to combinatorial polymers, such as nucleic acids and peptides, and, it generally requires knowing the sequences prior to the synthesis. Thus, the approach is not useful for complex molecules that cannot be synthesized and it is not effective without sequence information to guide synthesis. It does not lend itself to mixed arrays containing different kinds of immobilized reagents. The approach relies on reiteration of fairly complex steps and typically provides a low yield of array members in a high background of side products. Array members are formed in situ and cannot be processed, purified or assayed before use. The approach depends on high precision and reproducibility of complex synthesis steps, as well as uniformity of yield across a large number of differing products. Photo lithographic implementations require complex, high precision machinery and extremely high levels of skill, equipment and investment as does semiconductor manufacturing, from which it is partly derived. Photo lithographic approaches, thus, are as expensive as integrated circuit manufacturing, prohibitively so for many applications.

The array approach also is being applied to the discovery of new and useful inorganic materials, an approach spearheaded by Symyx, Inc. For instance, Symyx scientists combined thin film deposition and physical masking techniques adapted from the semiconductor fabrication industry to synthesize arrays (spatially addressable libraries) of solid state materials to screen for properties of interest. Arrays of inorganic compounds have been fabricated by this approach and screened for superconducting properties. These arrays contained up to 10,000 samples per square inch. Sample areas were squares as small as 200 micrometers on each side. (See Xiang et al., Science 268: 1738-1740 (1995) which is herein incorporated by reference in its entirety.) Similar arrays made by this approach were screened for large magnetoresistance. (See Briceno et al., Science 270: 273-275 (1995) which is herein incorporated by reference in its entirety.) More recently, a rare-earth phosphor of useful properties was discovered by combinatorial synthesis and parallel screening techniques using this approach. (See Danielson et al., Science 279: 837-839 (1998), which is herein incorporated by reference in its entirety). In this example, approximately 25,000 different compositions were defined on a surface by depositing constant or varying thicknesses of 5 oxides and 10 elements. The array was screened for UV photoluminescence. This method also relies on combinatorial build up of array members in situ (in the array) and, thus, it is not possible to assess quality of array members or to purify them before use. The method also requires resynthesis of all array members in each array. Therefore, like the synthesis approaches described above, it requires a very high level of precision and reproducibility. And the necessity to build up the array members individually in each array is more and more onerous as the number of array replicates increases, making it increasingly less practical as the number of arrays gets larger.

Thus, while the power of arrays is proven and it is clear that they will be breakthrough tools in many areas of research and development, existing techniques for making arrays both limit the types of array that can be made, the efficiency and economy with which they can be made, and the quality of the arrays and the ways in which they can be used. Clearly, all of the available ways of making arrays, and the arrays made by these methods, have undesirable limitations. None is suited for all applications and ways of making suitable arrays have not been developed for all applications.

Accordingly, there exists a need for ways to make arrays, for arrays, and for ways to use arrays that overcome these limitations. There is a need, therefore, for better methods and devices for making arrays, for improved arrays, and for improved methods and devices for using arrays. Thus, for example, there is a need for better methods and devices for making arrays, for improved arrays, for improved methods and devices for using arrays for determining physical, chemical and biochemical properties of samples, particularly for detecting and quantifying analytes in samples, such as molecular, macromolecular and cellular analytes in chemical, biological, veterinary, clinical, medical, forensic, agricultural, environmental, food, consumer, industrial and military samples, to mention just a few examples.

In sum, while the power of arrays is clear, current array-related technology has many shortcomings and limitations and better methods and devices for making arrays, improved arrays and improved methods and devices for using arrays are needed.

### ILLUSTRATIVE SUMMARY

It is therefore an object of the present invention to provide, among other things, novel and improved methods and devices for making arrays, novel and improved arrays, and novel and improved methods and devices for using arrays.

An object of the present invention is preferably to provide a method for making arrays of a plurality of array members, comprising the steps of: (A) providing a plurality of array members; (B) forming bundle members comprising the array members; (C) assembling the bundle members to form a bundle in which the array members are aligned; and (D) sectioning the bundle to produce wafers that comprise an array of the array members.

A further object of the invention is preferably to provide a method for making arrays for detecting a plurality of analytes, comprising the steps of: (A) providing a plurality of analyte binding reagents array members; (B) forming bundle members comprising of or comprising the array members; (C) assembling the bundle members to form a bundle in which the array members are aligned; and (D) sectioning the bundle to produce wafers that comprise an array of the analyte binding reagents.

Array members of the above-mentioned methods are, preferably, cross-sectioned perpendicular to their alignment, cross-sectioned at an angle of 10 to 80 degrees or 100 to 170 degrees to their alignment, cross-sectioned by a smooth planar cut, or cross-sectioned by a non-planar cut. Preferably, the surface area of such array members exposed by cross-sectioning is increased over that provided by a smooth, planar cut. Array members as mentioned are preferably comprised of or are disposed within a plastic, a glass, a metal or a ceramic. A plastic in accordance with such preferred methods can be polycarbonate, polyethylene, polymethylmethacrylate, polystyrene, a copolymer of polystyrene, polysulfone, polyvinylchloride, polyester, polyamide, polyacetal, polyethyleneterephthalate, polytetrafluoroethylene, polyurethane, or more preferably, polycarbonate, polyethylene, polystyrene, a copolymer of polystyrene, polysulfone or polyvinylchloride. Arrays of the above-mentioned preferred methods are preferably comprised of array members spaced about 1.0 to about 1,000 micrometers apart or having a cross-sectional area of about 1.0 to about 1,000,000 μm². Arrays preferably have a density of array members of about 250 to about 2,500,000 array members per square centimeter of cross sectional surface area of the array, about 10 to about 100,000 array members per square centimeter of total surface area at the assay, about 100 to about 2,500,000 aligned array members, or about 100 to 2,500,000 different aligned array members. In the above-mentioned methods, cross-sectioning preferably produces sections about 2.5 to about 2,500 micrometers thick.

An object of the invention is also preferably to provide a method for making arrays, comprising the step of cross-sectioning a plurality of aligned array members comprising at least two array members different from one another.

Another object of the invention is preferably to provide a method for making replica arrays, comprising repeatedly cross-sectioning a plurality of aligned array members to produce sections with at least one surface that exposes array members in the same disposition.

A further preferred object of the invention is to provide a method for detecting a plurality of analytes, comprising the step of cross-sectioning a plurality of aligne array members that comprise a plurality of analyte-binding reagents.

A further object of preferred aspect of the invention is also to provide a method for making replica arrays for detecting a plurality of analytes, comprising repeatedly cross-sectioning a plurality of aligned analyte binding reagent array members to produce sections with at least one surface that exposes array members in the same disposition, thereby replicating the array.

An object of the invention is to provide in any of the aforementioned preferred methods, preferably, analyte binding reagents that hybridize to DNA or RNA having specific nucleotide sequences; sequence specific binding reagents which are polynucleotides, peptide-nucleic acids or polyamides; sequence specific binding reagents which are oligonucleotides; analyte binding reagents that bind specific polypeptides; polypeptide-specific binding reagents which are polyclonal antibodies, monoclonal antibodies, a single chain antibody, or an antigen-binding fragment of an antibody; analyte binding reagents which are one or more of a nucleic acid, a polynucleotide, a DNA, an RNA, an oligonucleotide, a protein-nucleic acid, an aptamer, a ribozyme, a nucleic acid-binding polyamide, a protein, a peptide, a polypeptide, a glycoprotein, an antibody, an antibody-derived polypeptide, a receptor protein, a fusion protein, a mutein, a lipid, a polysaccharide, a lectin, a ligand, an antigen or a hapten. Any of the aforementioned methods can be are used to carry out an immunoassay, a hybridization assay, a ligand-binding assay or receptor-binding assay, or a substrate analog affinity assay.

A further object of the invention is to provide detection methods in accordance with any of the aforementioned methods, including preferably methods where analyte binding reagents are detected using radioactivity, fluorescence, phosphorescence or chemiluminescence.

It is an object of the present invention to provide devices comprising a plurality of preformed molecules or molecular complexes, or derivatives thereof, in a plurality of discrete and defined locations. It is a particular object of the invention in this regard to provide devices comprising preformed molecules or molecular complexes, or derivatives thereof, for determining analytes (i.e., that are analyte determining reagents, such as but not limited to, molecules, molecular complexes, derivatives or mixtures thereof).

The foregoing summary is not comprehensive of the invention in any respect. Rather, it describes very briefly certain specific objects and embodiments of the invention in order to provide an impression of the invention, albeit incomplete, that will facilitate a more comprehensive understanding based on reading the present disclosure as a whole, viewed in light of the knowledge of those skilled in the arts to which the invention pertains. The summary herewith presented does not portray the limits of the invention. Rather it is directed to particular embodiments aspects thereof. A full understanding of the invention is to be had only by careful consideration of the entirety of the present disclosure in light of the knowledge of those skilled in the arts to which it pertains.

### BRIEF DESCRIPTION OF THE FIGURES

The figures are provided to aid understanding of the invention herein disclosed. They portray certain specific illustrative embodiments and aspects of the invention. They do not portray the invention in its entirety in any respect and they do not portray limitations of the invention.

FIG. 1 illustrates the introduction of reagents into tubes. The figure shows the introduction of four reagents, numbered 1 through 4, into four empty tubes to produce four reagent-filled tubes. The reagents are indicated by patterns in the filled-tube lumen. The reagents exemplify array members, empty tubes exemplify structural members and the filled tubes exemplify bundle members, as those terms are used to describe the invention.

FIG. 2 depicts the assembly of eight reagent-filled tubes, numbered 1 through 8, into a flat ribbon. The ribbon provides an example of intermediate structures that may be useful in the invention.

FIG. 3 depicts the assembly of four ribbons, numbered 1 through 4, into a bundle in which the relative positions of the tubes remain the same. The illustrated bundle is comprised of bundle members (filled tubes), which are comprised of a structural member (tube) and an array member (reagent).

FIG. 4 depicts the production of wafers from a bundle. Four wafers are shown, each one at a different viewing angle. The wafers comprise an array of the reagents. Note that the disposition of the bundle members (filled tubes) aligns the array members (reagents) so that they form the desired array in the wafers made by sectioning the bundle.

FIG. 5 depicts several shapes and configurations of tubes.

(a) shows four configurations of hexagonal tube with a circular lumen: (1) shows reagent filling an untreated lumen; (2) shows reagent filling a surface-treated lumen; (3) shows reagent coating an untreated lumen which remains largely open, and (4) shows reagent coating a surface-treated lumen which remains largely open. The thickness of the surface treatment and reagent-binding layers are not to scale with the tube or lumen.

(b) shows several different shapes of tubes and configuration of lumen. The depiction is merely suggestive of the wide variety of tube and lumen shapes and combinations thereof useful in the invention.

FIG. 6 illustrates assembly of wafers into sub-assemblies and modules. The top of the figure shows a wafer with 400 array members in a 0.2 cm×0.8 cm, 10×40 array. The middle of the figure shows five wafers containing 2,000 array members in a 1.0 cm×0.8 cm sub-assembly. The bottom of the figure shows six sub-assemblies containing 30 wafers and 12,000 array members in a 2.0×2.4 cm module. The figure illustrates the build up of a large device from smaller units, which provides multiple ways to incorporate a given tube section into a device, and multiple chances to correct a faulty array member section in any one wafer or set of wafers.

FIG. 7 illustrates a device for contacting a wafer with samples and reagents for analysis. The device is shown from the top in a and from the side in b. It is shown from the side in c, joined to a reagent-delivery manifold. 1 is the wafer module. 2 is a sample delivery port. 3 is a reagent delivery port. 4 is a reagent exit port. 5 is the flow channel for sample and reagent, which runs from the sample and reagent entry ports directly over the wafer module to the reagent exit port. 6 is three areas for human and/or machine readable information on the device. 7 is sample being loaded into the sample loading port, covered by a septum (dark rectangle). 8 is a reagent delivery manifold with reagent delivery port 9 and reagent removal port 10. O-ring gaskets (black partial circles) provide a fluid-tight seal between the device and the manifold. Disposable “needles” in 9 and 10 puncture the septa of 2 and 4 to isolate the delivery channels of the manifold from the device.

### GENERAL DESCRIPTION

The present invention relates generally to methods of making arrays, to arrays, to using arrays, to devices for making arrays, and to devices for using arrays, among other things. In particular, the invention relates to producing arrays by sectioning a bundle comprising array members, to arrays thereby formed, to using these arrays, to devices for making arrays by bundling sectioning methods, and to devices for using the arrays formed by these methods, among other things.

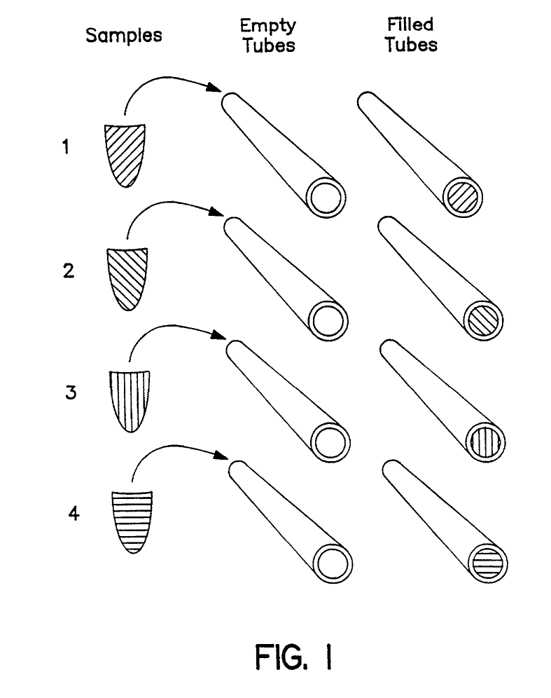

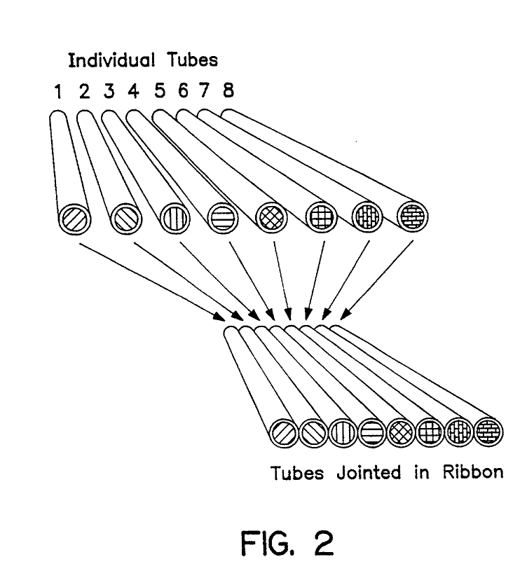

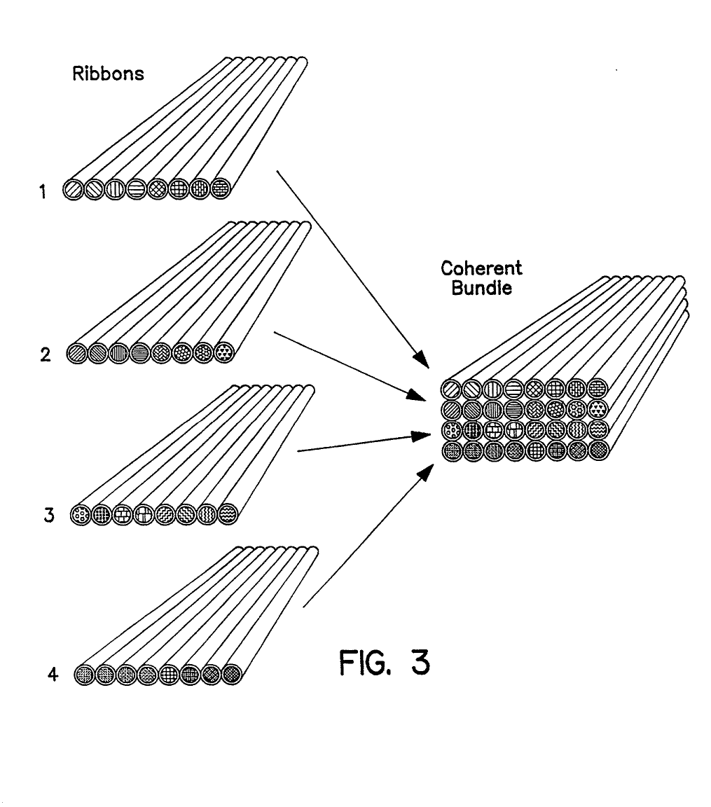

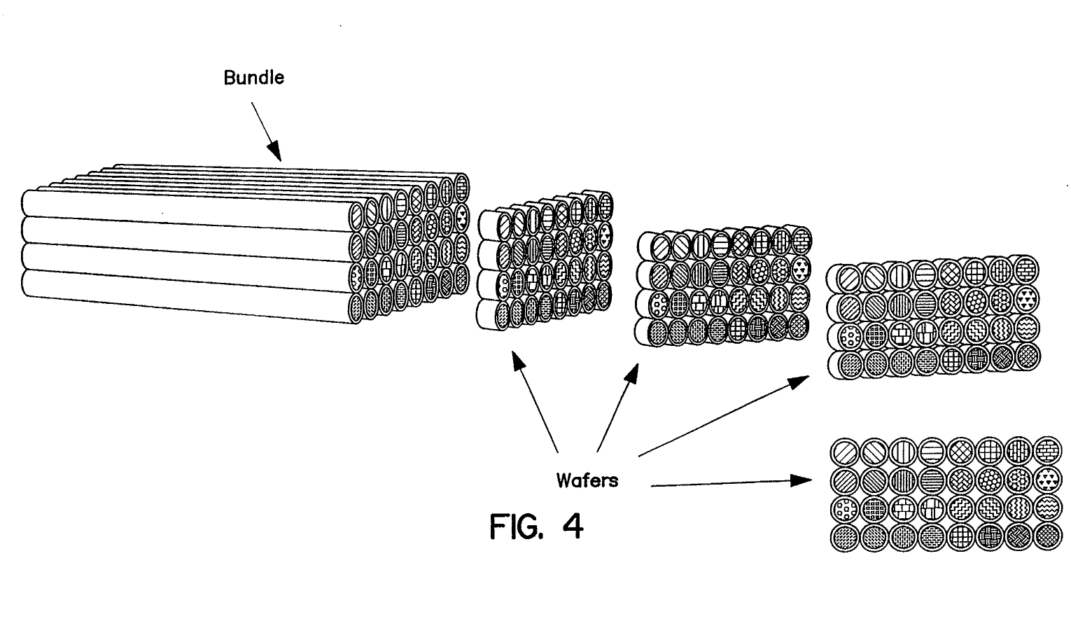

The following brief preliminary discussion is provided to facilitate understanding some aspects of the invention and terms used to discuss them, by way of reference to the specific embodiment of FIGS. 1, 2, 3 and 4. FIG. 1 shows four individual reagents introduced into four individual tubes to produce four filled tubes. The reagents, which will make up the array, are examples of array members. The tubes, which provide a structure for holding the liquid reagents, are examples of structural members. The filled tubes, which will be combined with other filled tube to form a bundle, are examples of bundle members. FIG. 2 shows the assembly of fibers into ribbons, illustrating the formation of an intermediate structure useful for forming bundles. FIG. 3 shows the assembly of ribbons into a bundle, illustrating the formation of a parallel coherent bundle in which the tube maintains the same position relative to other tubes throughout the illustrated length of the bundle. FIG. 4 shows the bundle again and four wafers produced by sectioning the bundle. Each wafer comprises the same array of reagents, and the figure illustrates the formation of identical arrays (in the wafers) by wafering a bundle. The filled tubes align the reagents in the bundle so that the desired array is produced when the bundle is wafered. In this regard, the filled tubes exemplify the use of bundle members to align the array members. Since, in the figure the array members (reagents) are disposed evenly within the illustrated bundle members (filled tubes), the bundle members are disposed identically throughout the bundle and the sections are cut identically, the array members are arranged identically in all the wafers in the illustration. In sum, the Figures illustrate a simple process of the invention for aligning array members in a bundle to produce arrays.

The invention, which is discussed in detail below, is not limited in any aspect to the particulars of the embodiments illustratively set out in FIGS. 1, 2, 3 and 4. Rather, the invention can be carried out with practically any shapes or sizes of array members and materials, and relates generally to any methods, arrays, and devices involving sectioning a bundle of aligned array members. General aspects of the invention in these and other regards, as well as many other particulars of specific embodiments, are described below. However, the discussion herein is necessarily illustrative, and a true understanding of the invention will be possible only by consideration of the disclosure as a whole from the point of view of those skilled in the arts to which it pertains.

ARRAYS

Generally, as to the invention disclosed herein, an array is an arrangement of array members. Often it is convenient to define an arrangement by positions of array members relative to one another in an array. A given arrangement may be defined in this way by the relative positions of some but not necessarily all of the array members in an array. In certain preferred embodiments, array members are in fixed positions in an arrangement.

In one general aspect, the invention is useful to produce replicate arrays all having the same arrangement of array members. In certain preferred embodiments of the invention, array members are disposed in the same arrangement relative to one another in all replicates of a given array. In certain highly preferred embodiments of the invention, the array members have the same fixed positions in all replicates of a given array. In other embodiments of the invention, some or all array elements in individual replicates vary in positions relative to one another. Where the arrangement varies between replicates, in preferred embodiments of the invention, the array members can be identified by other information.

ARRAY MEMBERS

Array members may be anything to be arrayed or arrayed. For instance, to give just a few examples, array members may be atoms, molecules, thin films, ceramics, glasses, metals, polymers, compounds, compositions, gels, mixtures, combinations of the foregoing and just about any other composition of matter. Preferred are those that have or are useful to identify or determine in other substances physical, electrical, magnetic, electromagnetic, chemical, biochemical, biological and other properties of interest. Examples of preferred embodiments in this regard include those that: bind analytes, absorb light, fluorescence, quench fluorescence, phosphoresce, those that are chemiluminescent, electroluminescent, sonoluminescent, piezoelectric, those that are polymers, metals, alloys, ceramics, organic compounds, inorganic compounds, biomolecules and biomaterials of interest, such as those to be screened for desired properties or those to be used as screening agents for properties of other substances, and combinations of any of the foregoing. Particularly preferred embodiments in this regard include polypeptides, including partial or complete proteins and peptides, polynucleotides, such as DNAs and RNAs, including relatively long polynucleotides and oligonucleotides, compounds that bind to polynucleotides sequence-specifically, such as peptide nucleotide acids and DNA sequence-specific polyamides, polysaccharides, ligands, ligand-binding biomolecules, molecules of pharmaceutical interest, chelating agents or those that bind to chelating agent-derivatives, fractions of cells or tissues, parts of cells or tissues, whole cells, whole living cells, derivatives and modified forms of the foregoing, and mixtures of any of the foregoing, to name just a few. In fact, arrays of the invention are not limited to any particular type of array member, and the foregoing examples, as well as the other examples set forth elsewhere herein are necessarily merely illustrative.

Particularly preferred in certain embodiments of the invention relating to binding assays, as described in greater detail herein below, are binding reagents, such as, but not limited to, DNAs, RNAs and other polynucleotides, polynucleotide-derivatives, such as PNAs and other polymeric compounds, such as certain polyamides, that bind to polynucleotides in a sequence-specific manner, antibodies and antibody-derived binding reagents, antigens, ligands, receptor polypeptides and derivatives thereof, aptamers, that bind specifically to cognate compounds or to cognate groups of compounds, such as DNA or RNA aptamers and polypeptide aptamers, to name just a few examples in this regard.

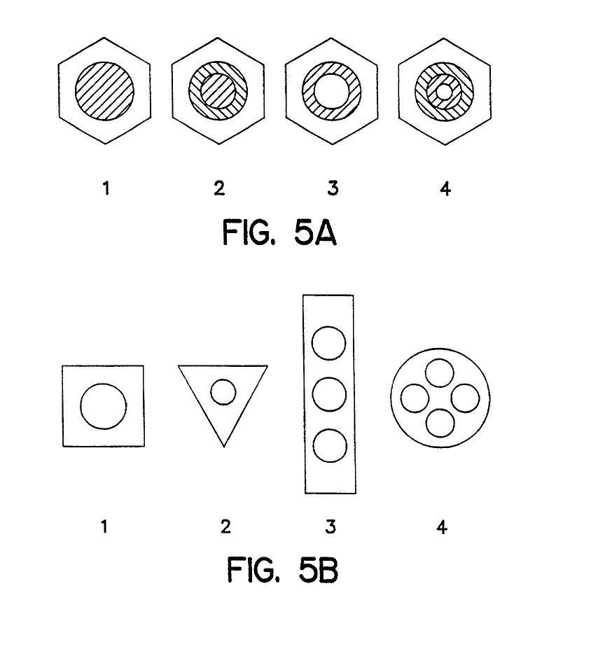

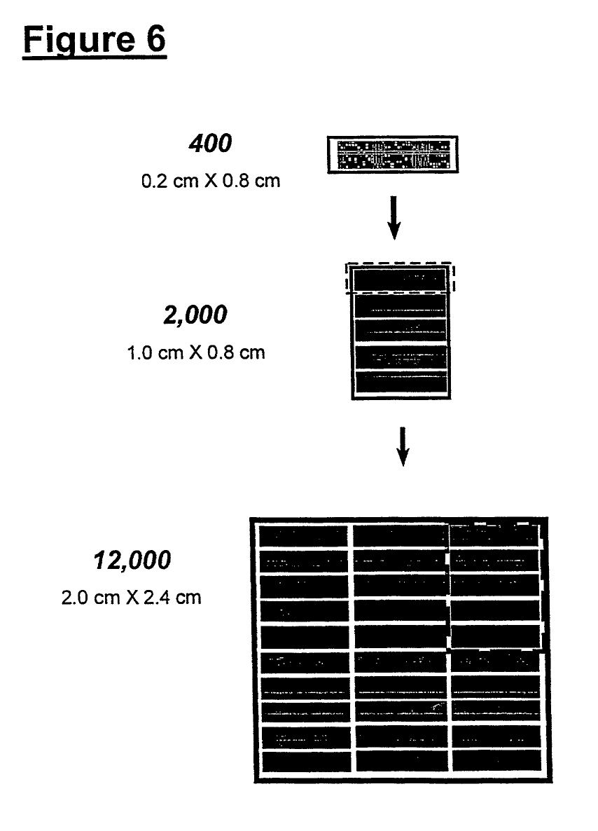

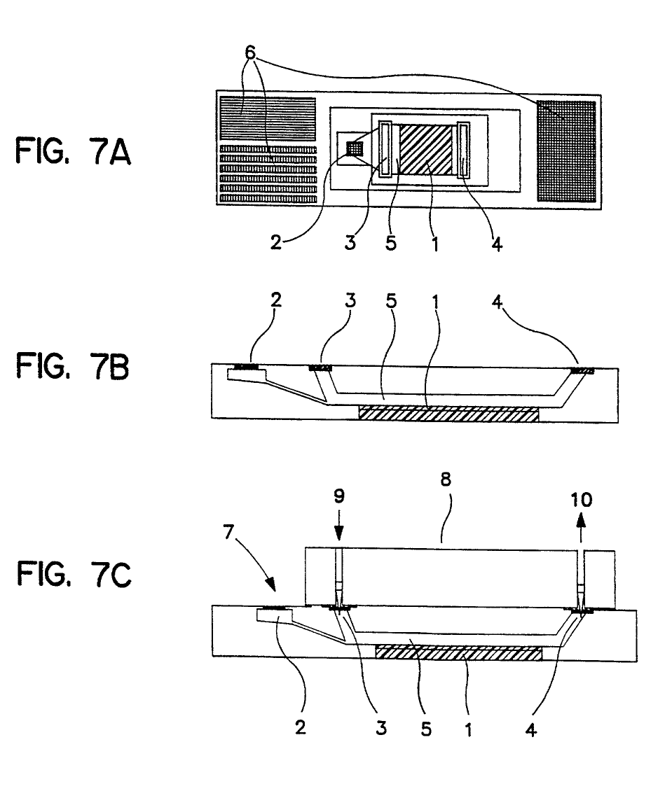

In general, array members may be any shape. For instance, an array member may be round, oval, ellipsoidal, triangular, square, rectangular, trapezoidal, pentagonal, hexagonal, octagonal, other regular or irregular polygon or any other regular or irregular shape. In preferred embodiments, the array members are uniformly shaped throughout the bundle. In other preferred embodiments the array members are homogeneous throughout the bundle. In particularly preferred embodiments the array members are both homogeneous and uniformly shaped throughout the bundle. For example, the array members in FIG. 4 are both homogeneous and uniformly shaped throughout the bundle. The same would be true for tubes having the cross-section shown in FIG. 5.

In some embodiments, such as those in which an array member itself serves as the structural member (see below for discussion of structural members) the array member may be shaped independently of a separate structural member. In other embodiments, an array member may be shaped by a structural member. Two illustrative specific embodiments in this regard are depicted in FIG. 5. The array members in the figure are distinct from the structural members. In 5(a)(1) and 5(a)(2) the array members are circular discs. In 5(a)(3) and 5(a)(4) the array members are annular with circular inner and outer perimeters. In all four examples the array members are disposed within a support having a circular inner perimeter and a hexagonal outer perimeter.

An array member may be formed independently of structural members. In addition, an array member may be formed within or on a structural member. Thus, for instance, to give but one particular example, an oligonucleotide array member may be synthesized, purified and characterized first and then loaded into a structural member, as depicted for the reagents in FIG. 1. An oligonucleotide array member also can be synthesized directly in a structural member. For instance, solid phase oligonucleotide synthesis can be carried out inside glass structural members to produce a bundle member comprising the glass structural member and an oligonucleotide array member attached inside the structural member. To mention just one other example in this regard, peptide array members also may be similarly synthesized on a structural member. In addition, array members can be attached to structural members in a precursor form and altered into final form after attachment.

Array members, such as those discussed, often cannot be formed into stable shapes for alignment and incorporation into bundles. Furthermore, many array members including many of the array members discussed above are useful only in small amount, and typically they are manipulated only in solution. It is desirable to provide these array members in a solution, suspension or mixture that can be solidified so that they can be formed into shapes for alignment and incorporation into bundles. This is particularly desirable when solid wafers are being produced and only the exposed surface of array members will be useful in the array. Thus, for instance, binding reagents, such as those discussed immediately above, can be dispersed in a gel precursor, such as, for instance, a solution of polyacrylamide and bis-acrylamide, introduced into a support, such as a tube and fixed therein by polymerizing the gel. Since polyacrylamide commonly is introduced into and polymerized in long narrow bore capillary electrophoresis columns (see below), and it is compatible with polynucleotides and polypeptides, it provides an apt example in this regard. A wide variety of other materials that can be gelled, polymerized or solidified can be useful in the invention in this regard.

Array members can have a wide variety of sizes and spacing, including but not limited to the following.

In preferred embodiments of the invention, for instance, there can be 10-100, 50-250, 200-800, 500-1,000, 750-2,500, 2,000-4,000, 2,500-7,500, 5,000-10,000, 7,500-15,000, 10,000-50,000, 25,000-75,000, 50,000-150,000, 100,000-300,000, 250,000-750,000, 500,000-1,500,000, 1,000,000-3,000,000, 2,500,000-7,500,000, 5,000,000-15,000,000 array members in an array.

Particularly preferred are 100-1,000, 1,000-5,000, 5,000-10,000, 10,000-50,000, 50,000-100,000, 100,000-500,000, 500,000-1,000,000, 1,000,000-10,000,000 and more than 10,000,000 array members in an array. Especially particularly preferred are less than 1,000, 1,000-10,000, 10,000-100,000, 100,000-1,000,000 and more than 1,000,000 array members in an array.

In preferred embodiments array members have cross-sectional areas of about 0.0025-0.0075, 0.005-0.015, 0.01-0.03, 0.025-0.075, 0.05-0.15, 0.1-0.3, 0.25-0.75, 0.5-1.5, 1.0-3.0, 2.5-7.5, 5.0-15, 10-30, 25-75, 50-150, 100-300, 250-750, 500-1,500, 1,000-3,000, 2,500-7,500, 5,000-15,000, 10,000-30,000, 25,000-75,000, 50,000-150,000, 100,000-300,000, 250,000-750,000, 500,000-1,500,000, 1,000,000-3,000,000, 2,500,000-7,500,000, 5,000,000-15,000,000 and 10,000,000-30,000,000 μm².

Array members can be spaced in arrays to suit a variety of applications. Preferably in many applications array members are spaced about 0.05-0.15, 0.1-0.3, 0.25-0.75, 0.5-1.5, 1.0-3.0, 2.5-7.5, 5.0-15, 10-30, 25-75, 50-150, 100-300, 250-750, 500-1,500, 1,000-3,000, 2,500-7,500 or 5,000-15,000 micrometers apart.

The density of array members in arrays preferably is about 10-100, 50-250, 100-350, 200-400, 150-750, 500-1,000, 750-2,500, 2,000-4,000, 2,500-7,500, 5,000-10,000, 7,500-15,000, 10,000-50,000, 25,000-75,000, 50,000-150,000, 100,000-300,000, 250,000-750,000, 500,000-1,500,000, 1,000,000-3,000,000, 2,500,000-7,500,000 or 5,000,000-15,000,000 array members per square centimeter of cross sectional surface area of the array.

In some preferred embodiments, such as flow through embodiments, the flat cross sectional surface area of arrays are much different than the total surface area. In preferred embodiments in this regard, preferred density of array members in the arrays is about 10-100, 50-250, 200-800, 500-1,000, 750-2,500, 2,000-4,000, 2,500-7,500, 5,000-10,000, 7,500-15,000, 10,000-50,000, 25,000-75,000, 50,000-150,000, 100,000-300,000, 250,000-750,000, 500,000-1,500,000, 1,000,000-3,000,000, 2,500,000-7,500,000 or 5,000,000-15,000,000 array members per square centimeter of total surface area.

Array members also can be of a variety of depths. In preferred embodiments array members are 0.1-0.3,0.25-0.75, 0.5-1.5, 1.0-3.0, 2.5-7.5, 5.0-15, 10-30, 25-75, 50-150, 100-300, 250-750, 500-1,500, 1,000-3,000 or 2,500-7,500 micrometers deep.

STRUCTURAL MEMBERS

Often array members are difficult to manipulate in the manner required for forming and using arrays in accordance with the invention. For instance, fluids do not hold their shape and cannot be formed into specific shapes. Fluid array members, such as solutions, thus generally must be put into a container to give them an appropriate shape and to align them. Structural member is the term used primarily herein for structures, such as containers, that provide support for array members, particularly so they can be aligned and assembled into bundles. Structural members can serve other purposes as well, such as providing additional support for bundle members or for bundles (see below), as alignment members in bundles and/or to facilitate assembly of bundles, as alignment members for edge to edge or face to face alignment or stacking of wafers and/or arrays or both, providing positional markers in arrays and/or wafers and/or providing informational elements in wafers and/or arrays, to name just a few.

Structural members can be made of a wide variety of materials, as discussed in the “Materials” section below.

In general, structural members may be any shape. For instance, an array member may be round, oval, ellipsoidal, triangular, square, rectangular, trapezoidal, pentagonal, hexagonal, octagonal, other regular or irregular polygon or any other regular or irregular shape. Structural members that have one or more lumen, such as tubes, are particularly useful for aligning array members and incorporating them into bundles. Structural members can have no lumen, one lumen, a few lumen, such as 1 to 10 lumen, or many lumen, such as 11 to 100 or 101 to 200 or 201 to 500 or 500 to 1,000 or more than 1,000. A very few illustrative embodiments in this regard are set out in FIG. 5(a) and (b), particularly 5(b). FIG. 5(a)(1) through 5(a)(4) shows a hexagonal structural member with a circular lumen. In 5(a)(1) the lumen is filled with an array member (exemplified by a reagent). In 5(a)(2) the lumen is coated and filled with an array member. In 5(a)(3) the array member is coated on the lumen wall, leaving the lumen of the structural member largely open. In 5(a)(4) the inner wall of the lumen is coated, the array member is layered on the coating, and the inner portion of the lumen remains open. 5(b) shows square, triangular, rectangular and circular structural members with one or more lumen into which array members can be introduced. As noted above, practically any shape can be used for structural members. The particular shape employed, the number and size of lumen, the disposition of any coating and of array members with respect to a structural member can be adjusted to best suit a given application and array.

Structural members also may be practically any size, typically dictated by the desired size of the array members in the array. Thus, in particularly preferred embodiments structural members have dimensional properties, such as cross-sectional areas, spacings, cross-sectional and total density and depth after wafering set forth above and elsewhere herein for array members. In particularly preferred embodiments, moreover, structural members have lumen that singly or in groups, provide array members with the aforementioned dimensions.

Structural members and array members generally are distinct from one another, but this need not be the case. Array members can provide their own support and, if so, they can be aligned and incorporated into bundles without structural members. However, more commonly, array members may be attached to or otherwise associated with or loaded onto or into structural members. For instance, the reagents, which depict array members in FIG. 1 are distinct from the round, hollow fibers which depict structural elements. This is but one particular type of embodiment of the invention, however. In other particular embodiments an array member may be integral to a structural element, such as an array member is dispersed in a structural member. In other embodiments of the invention, an array member itself is a structural member. Combinations of the foregoing are also useful in the invention.

BUNDLE MEMBERS

In preferred embodiments of the invention, a plurality of array members or a plurality of structural members comprising array members are grouped together to form a bundle. Each member of the bundle can be referred to as a bundle member. By way of example, in certain particular bundle member embodiments, for instance, an array and structural member are different and/or an array member itself also is a structural member and/or an array member is integral to a structural member. These and other types of bundle members may be used alone or in any combination in forming bundles. By way of illustration by reference to a specific type of embodiment, for instance, array members may be reagents and structural members may be round, hollow fibers, as depicted in FIG. 1. Bundle members are exemplified thus by the round, hollow fibers filled with reagents depicted in FIGS. 1, 2, 3 and 4.

BUNDLES

In accordance with a preferred embodiment of the invention, arrays are formed by sectioning a plurality of aligned array members or structural members associated with array members. In particularly preferred embodiments, the array members are aligned in a bundle. In certain very particularly preferred embodiments of the invention, the array members are coherently aligned in the bundle. Coherently aligned means, in this regard, that the array members are aligned in the bundle so that they can be identified and interrogated in arrays produced by sectioning the bundle. In particularly preferred embodiments in this regard, coherent means that array members are aligned in the same relative position with respect to one another in at least the portions of the bundle from which arrays are formed. In especially preferred embodiments in this regard, the members are aligned parallel with one another in the bundle. However, the alignment of array members in a bundle need not be parallel; many other arrangements can be used that provide for ready identification of array members in the array after sectioning.

A bundle may comprise both array members and other components. Other components include structural members, as discussed above, material for binding array members or structural members together and material for altering the properties of structural members or array members, such as light-reflecting or light-absorbing properties. A bundle also may comprise alignment members for aligning sections with one another, such as for horizontal or vertical alignment or stacking sections, or for mounting sections in a support or other type of device. Other components may be configured the same as, similar to or much different from the array members in the bundle. Bundle members may be assembled into bundles in final form or in an intermediate form. If the bundle members are in intermediate form, the bundle may be processed to provide bundle members in their final form for wafering. Processing the bundle in this regard preferably employs methods that preserve desired properties of array members in the array. One illustrative example in this regard is provided by a method for making arrays with nanochannel structural members in accordance with the invention.

WAFERS

Wafers, in accordance with the invention, comprise arrays and are produced from bundles. Generally, wafers are produced by cutting across a bundle, by which is generally meant sectioning a bundle at an angle to axis of alignment of the bundle members. In preferred embodiments, a bundle is sectioned so that all array members are disposed in the same way in the resulting wafers. Put another way, in preferred embodiments a bundle is sectional so that the arrays are the same in each of the resulting wafers. In particularly preferred embodiments in this regard the array members are in the same positions in the assays in each of the resulting wafers.

In a highly particularly preferred embodiment wafers are made by sectioning a bundle perpendicular to the axis of bundle member and array member alignment, much as illustrated in FIG. 4. In other preferred embodiments wafers are produced by sectioning a bundle at oblique or acute angles to the axis of alignment. Sectioning at an angle may be preferred where it is desirable to expose a larger surface area of array elements in the surface of the wafer. For instance, cutting the bundle in FIG. 4 at such angles would expose ovoid sections of each array member on the wafer surface, which would provide greater surface areas than the circular sections shown in the figure. Likewise, cutting a bundle at an acute or oblique angle to the alignment axis provides a larger wafer than cutting perpendicular to the alignment axis.

Wafers may be formed with planar faces, as illustrated in FIG. 4, and as discussed immediately above, or with any of a very wide variety of other shapes. For instance, wafers may be made with a v-shaped surface, a rippled surface, a grooved surface, a deeply trenched surface, and the like. In fact, wafers can be made in accordance with the invention with just about any shape, particularly those that can be made readily by shaping a cutting instrument and controlling its movement as it sections a bundle.

The faces of a wafer may be the same or different. Wafers with the same front and back faces can be made by using the same cut to section a bundle on both sides of the wafer. Wafer faces can be made different by using different cuts to section a bundle on each side of the wafer.

In much the same ways as described above for wafer faces, the wafer sides can be made symmetrically or asymmetrically in an equally wide variety of shapes, by, for instance, processing the sides of the bundle prior to wafering.

Wafers of any given shape, moreover, can be further processed to alter features of the faces or the sides or both.

Cutting, milling, drilling, forming, abrading, smoothing, pulling, extruding and other forming techniques well known to the fabrication arts, particularly techniques used to shape, form and finish metal, ceramic, glass and plastic can be employed in the present invention to produce and shape wafers to suit a given application.

MATERIALS

Virtually any material can be used for making arrays in accordance with the invention. The choice of materials for a given array will depend on details of the array members, how they will be incorporated or affixed in bundles, how the bundles will be assembled and processed, how the bundles will be sectioned for wafer production, how the wafers will be processed and how the arrays are to be used, to mention just a few considerations. Generally, where an array member is not itself the sole structural member of a bundle member or a bundle, there is greater freedom to choose material to be used for a structural member.

In general, materials suitable for use in the invention include any materials that can be shaped into desired form for structural and bundle members. Generally, materials that are relatively easy to form are preferred. Particularly preferred are materials that can be shaped to very high precision and very small feature sizes. Thus, particularly preferred materials possess the ability to form a desired configuration with specified dimensions and properties, including, but not limited to desired thickness and density (including having channels); the ability to load it with array members; and tolerance for various treatments including for instance, those associated with producing bundles and wafers and those associated with use of arrays. Among such particularly preferred materials are those that can be formed into long, narrow bore members, for example hollow fibers, that can hold array members and can be joined in bundles in accordance with the invention herein disclosed. Also preferred are materials that can be wafered conveniently, with high precision and very small geometries. Among particularly preferred materials, as well, are materials that are compatible with array members. That is, materials that do not deleteriously interact with or affect array members during the process of loading, bundling, wafering, storage, use or any other condition to which arrays may be subjected during assembly, production, storage, shipment or use. Additionally preferred materials are those that are compatible, in much the same regard, with downstream process steps and end uses. In one aspect in this regard, materials with a high degree of dimensional stability are preferred. In another aspect, materials resistant to solvents and materials used in downstream process steps and end uses are preferred. In another aspect in this regard, materials are preferred that facilitate detection, particularly for analyte detecting and/or determining applications of the invention.

Clearly, the invention can be practiced in too many ways to elaborate more than a few herein. Those skilled in the pertinent arts, having understood the invention, by taking into account such general considerations as those set out above and others that may be pertinent, should be able to choose appropriate materials of practically any sort that will be effective to carry out the invention in any particular application and circumstance. A few illustrative particulars are set out below as further guidance in this regard.

Particularly useful materials for making arrays in accordance with the invention include glasses, plastics, ceramics and metals. This is particularly the case for embodiments in which hollow fibers (of whatever exterior and lumen shape) are used to make arrays, because very long hollow fibers are readily fabricated from these materials and in many cases are commercially available. Moreover, particularly for glass and some plastics, many surface treatments and chemical derivations are well known that can be used to bind array members to structural members, for instance, or to bind structural members to one another, or to bind bundle members together to form bundles, to mention just a few. Similarly, many surface treatments and derivations are known for these materials that can be used for other aspects of the invention, such as detection.

Glasses are particularly preferred for the invention. A variety of well known fabrication techniques can be used to shape a glass into configurations for use in the invention. Likewise, a great many available glass formulations, treatments and chemical modification techniques readily can be employed in the invention. Thus, to mention just a few types of glasses: standard glasses, functionalized glasses and glass-ceramics all may be used in the inventions. Useful information in this regard is provided in The Biomedical Engineering Handbook, J. D. Bronzino, ed., CRC Press, 1995, particularly at pages 566-580, which is herein incorporated by reference in part pertinent to using glass in the invention.

More particularly, long glass hollow tubing is preferred for certain embodiments of the invention. Tubing of this type is commercially available in the form of gas chromatography (“GC”) column tubing, capillary electrophoresis (“CE”) column tubing and hollow fiber optic tubing. GC column tubing is available off the shelf, in length of several meters, with an outer diameter (“OD”) of 350 micrometers or less and with various inner diameters (“ID”) generally in the range of 50 to 200 micrometers for tubing with an OD of 350 micrometers. Generally, GC column tubing can be obtained with various inner wall coatings designed to facilitate GC analysis. Some of these coatings can be used advantageously to increase or decrease interactions of array members with the inner wall GC tubing used as a structural member, for instance. Other coatings may be useful for immobilization of certain types of array members. GC column tubing is available in a variety of glasses, with, as well as without, such coatings. Moreover, the processes used to make GC tubing are not limited to the aforementioned dimensions and can be used to custom fabricate tubing with other dimensions, including tubing that is meters or even hundreds of meters long with much smaller OD and ID.

CE tubing is available with dimensions similar to that of GC and also can be fabricated in a wide variety of other dimensions. CE tubing is particularly advantageous in one regard, since it often is designed to contain a polyacrylamide gel. The techniques developed for CE columns for delivering polyacyrlamide into CE columns and then polymerizing it to form a gel in situ are particularly in certain embodiments of the present invention in which an array member is introduced into a structural member in a solution that is then solidified in situ.

Hollow glass fiber optic tubing also is available with advantageous dimensions and properties. Glass pulling techniques for fiber optic tubing are highly advanced, and it is routine to manufacture single tubes several miles long. Although such fibers typically have solid cores, similar techniques can be used to produce very long, very narrow hollow fibers.

Useful plastics and/or polymers include, among a great many others, polycarbonate, polyethylene, methylmethacrylate, polypropylene, polyester, (poly)tetrafluoroethylene, (poly)vinylidenenedifluoride and the like. Many other plastics and polymers that can be used in the invention are well known to those skilled in the art, such as those described in, among other well known references, Modern Plastics, Encyclopedia '97, Volume 73, November 1996 and The Biomedical Engineering Handbook, J. D. Bronzino, ed., CRC Press, 1995 (in which, pages 581-610 are particularly useful in this regard), both of which are herein incorporated by reference in their entirety in parts pertinent to using plastics and/or polymers in the invention.

A great variety of plastic fiber and tubing is available for use in the invention. Readily available polyethylene (“Intermedic”) tubing is available in several lengths with an OD less than 500 microns. Hollow plastic fiber optic tubing also is available, Moreover, plastic of desired dimensions, such as tubing of particular length, OD and ID, generally can be custom fabricated at reasonable cost to just about any size and configuration.

Similarly, ceramic and metal tubing for use in the invention can be obtained commercially in off the shelf dimensions or can be custom fabricated.

ASSEMBLING COHERENT BUNDLES

Technology developed for manufacturing image pipes can be adapted to and provides guidance for the assembly of coherent bundles in accordance with the invention. Image pipes are bundles of optical fibers that transmit an image from one end surface to the other. The optical fibers in the pipe are arranged “coherently,” which is a term of art meaning that fibers maintain the same position relative to one another in the cross-section everywhere along the length of the pipe.

As noted elsewhere herein, optical fibers per se consist of a core and a cladding, both solid. Fibers can be made of silicon glass or certain plastics. Fiber of submicron diameter can be mass manufactured. Hollow fibers can be manufactured by similar techniques either directly or by forming fibers with a core that can be removed. Some methods for making fibers, particularly from glass, are set out in Hecht, UNDERSTANDING FIBER OPTICS, Sams Division of Prentice Hall, Carmel, Ind. (1987), which is incorporated herein by reference in its entirety relating to manufacture, coating, mating and principles of fiber and fiber optic manufacture and use. Generally, as noted elsewhere herein hollow core optical fibers are preferred.

One familiar use of light pipes is in arthroscopic surgical devices to pump an image from inside the body to the operating physician. The same techniques used to assemble individual optical fibers in perfectly parallel arrangement also may be used to assemble fibers of other types in this fashion. However, techniques for making light pipes that involve pulling arrays of glass tubes down to a narrow bore typically involve high temperatures that often are not compatible with array members.

PROCESSING BUNDLES PRIOR TO WAFERING

Some preferred embodiments involve processing a bundle prior to wafering. In a particularly preferred class of such embodiments bundles are processed prior to wafering to alter the dimensions of array members. In embodiments of this type, bundles are made of materials that can be pulled (stretched, elongated), preferably repeatedly, preferably to form extremely small channels. It also is preferable that materials allow the bundles to be divided into sections, particularly after pulling and, preferably, sections from the same or different bundles joined or fused to one another along their sides, so that many bundle members can be pulled in concert. A group of these filled bundle members are aligned and affixed in a bundle. The bundle is pulled, without changing total volumes, so that the bundle members are greatly elongated and greatly decreased in cross section. This process is repeated, combining the same or different bundles after pulling, to form bundles of desired geometry. For instance, a circular bundle member 1 cm in diameter and 10 centimeters long can be pulled to 100 times its length to form a bundle member 0.1 centimeters in diameter and 10 meters long. 100 such pulled bundle members, each containing a different array member can be aligned in a bundle. This bundle can be divided into 100 equal bundles each 10 centimeters long. When pulled to 100 times its length each of these 10 centimeter long bundles will form a bundle 10 meters long in which each bundle member is 100 microns in diameter. 100 such bundles each containing different array members can be joined into a single bundle containing 10,000 different array members in 10,000 different bundle members each about 100 microns in diameter. Sectioning all 10 meters of this bundle into wafers 150 microns thick, with 50 microns waste between wafers, can provide 50,000 wafers about 150 microns thick, each containing 10,000 array members in 10,000 bundle members 100 microns in diameter. Forming and sectioning all 100 of the equal bundles in the same way can provide a total of 5,000,000 wafers. Thus, 10,000 bundle members originally 10 centimeters long and 1 centimeter in diameter can provide 5,000,000 wafers each containing replicate 10,000 member arrays.

One embodiment in this regard, particularly useful to make arrays with lumen, such as flow through arrays, utilizes two materials. One is resistant to and the other is degraded by a process herein referred to as an excavating process. Array members are immobilized on the lumen wall of support members formed of the resistant material. The lumen then are filled with the material that is degraded by the excavation treatment. Once inside the lumen the material is solidified. Pulling, sectioning and wafering is carried out as described immediately above, with the same effect. Wafers produced from the pulled bundles then are subjected to the excavating process, which degrades the fill material and reopens the lumen, now of much reduced cross-section. A technique of this types is used to make nanochannel glass wafers that can be used in flow-through biochips, such as those being developed by Gene Logic. However, in that case, the nanochannel glass is manufactured first and then patterned with oligonucleotide probes. The glass manufacturing process involves treating glass wafers with very strong acid to remove acid-sensitive glass core material and form nanochannels. The acid is not compatible with many array members, such as oligonucleotides. Excavating treatments in these embodiments, therefore, preferably are compatible with the array members and intended uses of the arrays. Excavating processes that can be useful in this regard include light mediated degradation of light-degradable plastic.

SECTIONING BUNDLES TO FORM WAFERS

Wafers can be cut from bundles using any of a wide variety of methods, in a very wide variety of ways. As noted elsewhere herein, for example, the attack of the cut can be perpendicular to the bundle or it can be at a different angle. Cuts can be symmetrical or asymmetrical, both with respect to the bundle itself and/or with respect to the two sides of the wafer. Asymmetrical cuts, particularly of the latter type, are useful, for instance, especially when the faces of a wafer are desirably different. Cuts can be planar (flat) or they can be other shapes. Particularly preferred in this regard, for example, in addition to planar cuts, are cuts that increase or facilitate exposure of array elements desirable in an end use, such as saw tooth cuts that deeply corrugate array members thereby increasing their exposure. Cuts can be complete in themselves or can be compounded with one another to produce more complexly shaped sections, as well. These are but a few of the variations possible in this regard.

Many types of cutting methods can be used to section bundles to produce wafers and arrays in accordance with the invention. A number of parameters generally should be considered in choosing the method to use in a given application and circumstance. First, the cutting method should be effective for all the materials in the bundle, including array members, any structural members and any other components of the bundle that are to be sectioned. The cutting method should be compatible with the bundle components, particularly the array members and any other components important to further processing or using the wafer and the array being produced. Methods that minimize waste are particularly preferred. Thus, where mechanical cutting is employed, methods that minimize kerf are preferred in this regard. Methods that minimize contamination are preferred as well. To produce wafers from open bundles (i.e., bundles with openings in cross section) cutting methods that do not foul or close the opening are preferred. Generally, preferred cutting methods also minimize environmental stress, such as heat, exposing the bundle and wafers to abrasives or to lubricants, solvents, coolants or the like, especially stresses that may deleteriously effect array members or functional aspects of arrays and/or wafers. The following discussion of more specific cutting methods further illustrates this aspect of array production in accordance with the invention. In general, methods that can cut with greater precision are more highly preferred than less precise methods. In particular, for many applications and embodiments, the thinner the sections that can be produced the better. In this regard, low vibration, positionally stable cutting methods are preferred.

Mechanical methods for cutting, among other things, glass, plastics, ceramics and metals are well known. Such cutting devices that can be used to section bundles to produce wafers include knife-edge devices. Microtomes are a notable of the type of knife edge device that can be used to section bundles to provide very thin sections. Microtomes are designed to produce serial thin sections of tissue, typically 1 to 10 micrometers thick. Commercially available microtomes designed for sectioning fixed materials are useful in some embodiments of the invention in this regard. Likewise, cryostats (microtomes designed to cut frozen material) can be used to cut bundles in some embodiments of the invention. Both types of microtomes are particularly advantageous for sectioning bundles, when they are compatible with the bundle materials. They provide smooth cuts and surfaces. Generally, microtome cuts do not produce discemable contamination across the cut surface. The cuts can be made without distorting shape. Serial cuts are uniform. Spacing of cuts can be controlled very precisely and accurately. The cuts do not produce waste. They do not generate heat and they do not require lubricant, solvents or cleaning solutions. They can be readily automated to cut through a bundle continuously and to place each wafer in turn onto a carrier or other device, as desired.

Ultra high pressure liquid cutting is another useful technique for sectioning bundles to produce wafers in accordance with the invention. Ultrafast, jet streams of liquid, such as those used to cut steel and other metals, provide a clean, precise, temperature controlled cut that can be quite useful in some embodiments of the present invention. Methods of this type are particularly preferred for cutting glass, metal and ceramics, particularly when microtomes cannot be used.

Laser cutting also is a preferred cutting technique in certain embodiments of the invention.

Other mechanical cutting devices that may be useful in the invention include rotary and reciprocating cutting devices, including circular saws, band saws and wire saws, to name just a few. Tools of this type can be used in the invention, in keeping with the foregoing considerations.

The foregoing list is merely illustrative. Clearly, a great many other techniques, well known to those of skill in the pertinent arts, can be used to section wafers in accordance with the foregoing requirements.

ILLUSTRATIVE EXAMPLES RELATING TO ANALYTE ANALYSIS

The following discussion illustrates various aspects of the invention, by reference to certain specific embodiments useful to detect and, in some cases quantify, analytes in samples. The discussion is merely illustrative and discloses general features of the invention solely by way of specific examples. The invention is not limited in any way to particulars or details of these examples. For instance, the invention is not limited to analyte detection or quantification using analyte binding reagents, as is the discussion in these examples. Likewise, the invention is not limited to the particular assays or array formats discussed below. Rather the examples merely exemplify a few preferred embodiments of the invention and thereby illustrate aspects of its more general features.

Detecting and quantitating substances of various sorts in a wide variety of samples are important components of many economically important activities. For instance determining analytes, such as molecular, macromolecular and cellular analytes is important in biological, chemical, veterinary, clinical, forensic, agricultural, food, environmental, consumer products, process stream, quality control, military-related and other types of samples. Arrays, array-based devices, and array-related methods and apparatuses can be used for analysis of analytes in all of these types of samples, among others. Analyte determining arrays can be used in this regard for, to name just a few examples, clinical and veterinary diagnostic analyte analysis, forensic analysis, food quality monitoring, agricultural monitoring, environmental monitoring, monitoring of microbial agents, chemical and biological warfare agent monitoring, and process control monitoring. The following discussion illustrates the use of the invention in regard to specific embodiments of analyte-determining arrays.

NOMENCLATURE IN THE FOLLOWING DISCUSSION

A variety of terms are used in the following discussion in ways specifically relating to the exemplified analyte-determining embodiments of the invention. The following discussion provides illustrative explanations of some of the terms, as an aid to understanding the discussion and, thereby, the invention. However, the brief discussion immediately below does not provide exhaustive definitions, and it is not intended to circumscribe limitations of the invention, which can only be understood from careful consideration of the disclosure as a whole in light of related and ancillary knowledge in the arts to which it pertains.

Sample

Samples suitable for analysis in the present invention include any sample that can be brought into contact with assays in a chip for effective detection or quantification of an analyte. Preferred samples are homogeneous liquid samples. Included are biological fluids such as blood, serum, urine, saliva, spinal fluid, tears, lymph, bile, peritoneal, and wound fluids. Also included are homogenized biological samples such as homogenized mucous and homogenized bowel samples, as well as homogenized biopsy samples. Preferred samples include the aforementioned biological fluids, of which blood, serum, urine and salvia are particularly preferred.

Analyte

In the following discussion, any molecule, compound substance, organism or other thing that is to be detected or quantified is referred to as an analyte.

ABR

In the following discussion, a reagent that binds to and is useful for detecting, quantifying or analyzing one or more analytes is referred to as an analyte binding reagent (“ABR”). Generally, an ABR is any entity that binds to and can be used to detect the presence or determine the amount of an analyte. An ABR and the analyte(s) it binds are referred to as cognates. In some preferred embodiments the ABRs bind their cognate analytes very tightly and with great specificity. In other preferred embodiments, ABRs bind their cognate analytes much less tightly and with much less specificity. Such lower affinity and specificity ABRs are particularly useful in arrays that implement nose-like sensing paradigms. Analytes can be detected and quantified by binding to one or to more than one ABR.

Molecule, Compound, etc.

It will be appreciated that any atom, molecule, complex of molecules, mixture of molecules or molecular complexes, molecular aggregates, macromolecular or other complexes, aggregates, combinations of the foregoing or any other assemblage of matter can be used in accordance with the present invention if it is useful to the determination of any other assemblage of matter (as illustrated by the foregoing illustrative listing) and it can be incorporated into the devices of the invention or used in the methods and apparatus of the invention herein described. The terms molecule and compound and related terms generally are used herein to refer to any of these.

Chip

As used below, chip means the same as wafer, and as unit, unit surface and dice.

Element

Usually the smallest feature on a chip is referred to as an element. Most often it constitutes a defined area that contains a particular analyte binding reagent. The signal from an element may be obtained by sampling and calculation. Thus, for instance, a laser (or other sensing element) for reading an array can be smaller than the array elements, in which case the smallest signal-generating area is defined by the laser, not the array.

Assay

The part or parts of a chip relating to a given assay or test is referred to as an assay or assay areas. An assay may comprise one or more elements, which may be grouped or distributed in an array. For instance, an assay may comprise a graded concentration of an ABR to provide a quantitative curve of analyte binding. An assay also may comprise negative and positive controls.

Autoanalyser

An instrument for automatically processing sample test matrixes, generally comprising (1) a mechanism to transport carriers through the instrument, (2) a microprocessor controlled liquid delivery system that delivers appropriate reagents to the test matrix according to its type, (3) a system for digitizing all the assay results within each element in the matrix, and (4) a computerized analysis system to determine the results of each assay, to report the results, and to bill for the requested tests.

GENERAL CONSIDERATIONS FOR ANALYTE ANALYSES USING ARRAYS

The nature of the ABRs, the analytes and the sample(s) in which the analytes are to be determined largely will determine the analytic chemistries that are used in a given type of assay of the invention. Those of skill will appreciate from the present disclosure how to adapt particular assay systems for use in the present invention. Many analytic chemistries have been described and are well known to those of skill in the art that can be usefully employed in accordance with the present invention. These are described broadly in, for instance, BIOSENSORS, AN INTRODUCTION, Wiley and Teubner, Chichester and Stuttgart (1996), HANDBOOK OF BIOSENSORS AND ELECTRONIC NOSES, MEDICINE, FOOD, AND THE ENVIRONMENT, Erika Kress-Rogers, ed., CRC Press, Boca Raton (1997) and IMMUNOCHEMICAL ASSAYS AND BIOSENSOR TECHNOLOGY FOR THE 1990S, Nakamura et al., eds., American Society for Microbiology, Washington, D.C. (1992), each of which is herein incorporated by reference in its entirety in parts pertinent to analyte analysis procedures and chemistries. Numerous other publications, such as those cited in the forgoing references, set out assay procedures in much greater detail, and are well known to those of skill in art.

The invention provides several features that will be found advantageous in most, or all, types of assays. First, the invention provides a method to analyse many different analytes in a sample all-at-once. As set forth above, the method utilizes very small amounts of ABR to analyse a sample. In the most highly preferred embodiments, very small amounts of sample are used. And where reagents are employed, the most highly preferred embodiments of the invention use very small amounts of these reagents as well.

Similar considerations apply to the sample.

It will be convenient in many cases to employ an autoanalyser to carry out assays.

An alternative to liquid or gas reagents delivered by an autoanalyser may be preferred in low use and/or remote environments. In these situations development reagents may be provided as part of the platform which will incorporate a device for controlling delivery of the reagents to the assays effective for properly developing results. In a preferred embodiment of this type the reagents are in a gel in a geometry that provides reagent assay contact effective for proper development when the gel is extruded unidirectionally across the assays following appropriate exposure to sample. It will be appreciated by analogy that the operation of this embodiment of the invention is akin to the self-developing films that have been marketed by Kodak and Polaroid, among others, although the analogy should not be taken too literally.

STANDARDIZATION

It will be appreciated that there may be differences between the assays in an array. Therefore, each assay in an array may be designed to perform well under certain specified conditions to be employed. For instance, the assays in a given array might all desirably be designed to work well with a given autoanalyzer of limited flexibility and narrow dynamic range of detection. There are many ways to achieve desirable operative similarity of array assays in accordance with the invention, a few of which are described below.

The amount of ABR can be adjusted for each assay to work well within the available dynamic range. In addition, the amount of a detecting reagent or the amount of label in the detecting reagent may be adjusted. For instance, in a sandwich antibody assay, the amount of first antibody immobilized on the surface may be varied for each assay to work well with the expected sample in the available protocols and detection regimes of the analyzer. The amount of second antibody that binds to the antigen also can be controlled, by its concentration in the second antibody mix. Also, the degree of labeling of a detection reagent, such as a fluorescent tag or biotin affinity label on a secondary antibody, can be adjusted to help insure that the signal from each assay is within the working range of the analyze. For example, by reference to sandwich assays using biotinylation, since each first antibody may be separately immobilized, each second antibody can be separately biotinylated, and many assays can be tested simultaneously in a given autoanalyser protocol. It will almost always be possible to adjust the parameters of any particular assay to a standard protocol.

ILLUSTRATIVE ANALYTE ASSAY METHODS

The invention is useful for, among many other things, carrying out any assay predicated on binding of one member of a binding to the other, such as binding of an analyte binding reagent and cognate analyte. Numerous assays have been described and are well known to those of skill in the art that can be usefully employed in accordance with the present invention. Many such techniques are set out in some detail in, for instance, BIOSENSORS, AN INTRODUCTION, Wiley and Teubner, Chichester and Stuttgart (1996), HANDBOOK OF BIOSENSORS AND ELECTRONIC NOSES, MEDICINE, FOOD, AND THE ENVIRONMENT, Erika Kress-Rogers, ed., CRC Press, Boca Raton (1997) and IMMUNOCHEMICAL ASSAYS AND BIOSENSOR TECHNOLOGY FOR THE 1990s, Nakamura et al., eds., American Society for Microbiology, Washington, D.C. (1992), each of which is herein incorporated by reference in its entirety in parts pertinent to assay methods.

In certain preferred embodiments of the invention, immunological reagents are used, particularly antibody-derived reagents. In other preferred embodiments, oligonucleotide and/or PNA reagents are used. Other preferred embodiments relate to organic affinity reagents (ABRs), such as polyamides that can bind DNAs sequence-specifically. Still other preferred embodiments relate to random “sequence” polymers, such as random sequence DNAs, screened for analyte-binding ability and then used as ABRs.

Some preferred embodiments of the invention relate to high affinity and specificity ABRs, while other preferred embodiments relate to lower affinity and lower specificity ABRs. Particularly preferred regarding low affinity and/or low specificity ABRs are embodiments of the invention in which analytes are determined by patterns of binding to sets of ABRs. Still further preferred embodiments relate in this regard to combinations of ABRs with differing specificity, affinity and avidity for their cognate analytes. Particularly preferred embodiments use information from a variety of such ABRs to determine an analyte.

Illustrative analyte assay methods for use in the invention are described below. However, the illustrative methods are only a very few among many well known assay paradigms that can be practiced in the invention.

Sandwich Assays

In a highly preferred embodiment, sandwich assays are used in the invention. In certain highly preferred embodiments in this regard, first antibodies specific for cognate antigens are immobilized in an array, in accordance with methods described herein above. The array is exposed to a sample under conditions effective for antibodies in the array to bind cognate antigens in the sample. Thereafter, the array is washed and then exposed to second antibodies that bind to the cognate antigens under conditions effective for the secondary antibodies to form Ab1-cognateAg-Ab2 sandwiches with cognate antigens bound to first antibodies in the array. The formation of the sandwiches then is detected, thus detecting the presence of the cognate antigens in the sample.

Detection can be qualitative or quantitative. Generally, the second antibodies in sandwich assays are detected independently of the first antibodies. Thus, the first antibodies might be mouse monoclonals and the second antibodies might be goat polyclonals. More conveniently, first and second antibodies both may be mouse monoclonal, but all the second antibodies will have been covalently bonded to a detectable label, such as a fluorescent label. Other labels, of which many are know to those of skill in the art, can be used. Non-diffusing labels are preferable in some situations, where diffusion would preclude accurate localization, for instance. However, solution ELISAs also can be used in some embodiments.

Alternatively, second antibodies may be attached to a chemical hook which subsequently attaches the detectable label. The well known avidin-biotin system can be used in this way. For instance, the secondary antibodies can be tagged with biotin, a fluorescent label can be attached to avidin, and the presence of cognate antigens can be detected by fluorescence of labeled avidin that binds to biotinylated secondary antibodies in Ab1-cognateAg-Ab2 sandwiches formed on the array.

Those skilled in the art will appreciate the wide variety of immunoassay paradigms and the large number of detectable labels that can be used in much the same way as discussed above for sandwich immunoassays and fluorescence or ELISA detection. A general description of many such techniques that can be used in accordance with the present invention is provided in PRINCIPLES AND PRACTICE OF IMMUNOASSAY, 2nd ed., Price and Newman, eds. Stockton Press, New York (1997), which is incorporated herein by reference in its entirety.

Hybridization Assays

Also particularly useful in the invention are hybridization assays and methods for detecting hybridization. Such assays can take a wide variety of formats and are well known to those of skill in the art. Particularly preferred for use in accordance with the present invention are assays that work well in a solid-phase format in which a capture probe is immobilized on a solid phase, such as a surface, and localizes specific hybridization thereto. A number of formats, assay parameters to consider and detectors for chip-based hybridization assays are discussed by Titball and Squirrell in Probes for Nucleic Acids and Biosensors, Chapter 4 in HANDBOOK OF BIOSENSORS AND ELECTRONIC NOSES, MEDICINE, FOOD, AND THE ENVIRONMENT, Erika Kress-Rogers, ed., CRC Press, Boca Raton (1997), which is incorporated by reference herein in its entirety. Several formats for hybridization assays are illustratively discussed below; but, many other approaches also can be employed in accordance with the invention.

One approach to hybridization assays is direct detection of duplex formation. Several methods for so-doing are well known to those of skill in the art. Further, several such methods have been implemented in microchips by companies such as Affymetrix and Hyseq. In such approaches polynucleotides in the sample are labeled and their hybridization to the probe polynucleotides (or PNAs) on the chip are detected directly. The labels generally are fluorescent in common implementations of this approach; but, many other types of label can be used, as discussed elsewhere herein.

Another approach to such methods is based on primer elongation, exemplified by the following method for simultaneous analysis of a plurality of polynucleotides in a sample, which comprises the steps of: (A) providing a surface having immobilized in a predetermined pattern thereon a plurality of first oligonucleotide probes stably hybridizable specifically to one or more polynucleotides, wherein the first oligonucleotide probes provide 3′ hydroxyl groups for elongation, and wherein each position in the predetermined pattern exclusively contains a first oligonucleotide probe or mixture of probes specific for a polynucleotide or mixture of polynucleotides of the plurality of polynucleotides; (B) contacting the surface with a sample under conditions effective to hybridize polynucleotides in the sample specifically to the first oligonucleotide probes or mixture of probes immobilized on the surface; (C) removing from the surface components of the sample other than polynucleotides specifically hybridized to the immobilized first oligonucleotide probes on the surface; (D) contacting the surface with a solution comprising a polymerase for primer extension and a detectably labeled polymerase substrate under conditions effective to extend hybrids formed by the probes and polynucleotides of the sample, thereby detectably labeling the hybrids immobilized on the surface; (E) removing from the surface the components of the solution other than the labeled hybrids immobilized on the surface; and (F) determining the detectable label in each position of the pattern on the surface, thereby determining the polynucleotides in sample.

In certain preferred methods of this type the detectable label is a fluorescent label. In other preferred embodiments other types of labels are employed. Also preferred are mass tags for mass spectral detection and analysis. Also preferred in certain embodiments of this aspect of the invention are methods wherein the polymerase is a DNA polymerase and one, two, three or all four of the deoxyribonucleotide triphosphates are detectably labeled with a fluorescent label. In other preferred embodiments in this regard the polymerase is a reverse transcriptase and one, two, three or all four of the deoxyribonucleotide triphosphates are detectably labeled. In certain preferred embodiments in both regards, fluorescent labels are preferred. In another regard, certain preferred embodiments are those in which the Tₘ or T_{d} of duplexes formed by hybridization of sample polynucleotides to immobilized probes are within 1, 2, 3, 4, 5 or 10 degrees centigrade of one another. Particularly preferred are those in which the duplexes are within 1, 2 or 3 degrees centigrade under the conditions used for hybridization.

Also useful in the invention are methods for simultaneous analysis of a plurality of polynucleotides in a sample, comprising the steps of: (A) providing a surface having immobilized in a predetermined pattern thereon a plurality of first oligonucleotide probes stably hybridizable specifically to the polynucleotides, wherein the first oligonucleotide probes provide 3′ hydroxyl groups, and wherein each position in the predetermined pattern exclusively contains a first oligonucleotide probe or mixture of probes specific for a polynucleotide of the plurality of polynucleotides; (B) contacting the surface with a sample under conditions effective to hybridize polynucleotides in the sample specifically to the first oligonucleotide probes immobilized on the surface; (C) separating from the surface components of the sample other than polynucleotides specifically hybridized to the immobilized first oligonucleotide probes on the surface; (D) contacting the surface with a plurality of detectably labeled and ligateable second oligonucleotide probes stably hybridizable specifically to the polynucleotides, wherein the second and the first oligonucleotide probes are ligateably adjacent when hybridized to a polynucleotide from the samples; (E) separating from the surface, components of the sample other than second oligonucleotide probes hybridized to the polynucleotides hybridized to the immobilized first oligonucleotide probes immobilized on the surface; (F) contacting the surface with a ligase for ligating the ligateably adjacent first and second oligonucleotide probes hybridized to the polynucleotides under conditions effective for ligating the first and second oligonucleotide probes; (G) separating from the surface detectable label other than detectable label in second oligonucleotide probes ligated to the first oligonucleotide probes immobilized on the surface; and (H) determining the detectable label in each position of the pattern on the surface, thereby determining the polynucleotides in sample.

In certain preferred methods of this type, step (G) additionally comprises melting the polynucleotides hybridized to the first and second oligonucleotide probes, whereby detectably labeled second oligonucleotide probes ligated to first oligonucleotide probes remain bound to the surface via the first oligonucleotide probes immobilized on the surface in the predetermined pattern, and the other polynucleotides are removed.

In still another approach to hybridization assays, the immobilized probe contains two labels that interact through an energy transfer mechanism that is affected by duplex formation. The energy transfer is detectably altered by probe-target binding in a way that enables analyte detection and determination. For instance, the two label can exchange energy by Forster energy transfer. Forster energy transfer efficiency is proportional to the inverse sixth power of the distance between the two labels. Accordingly, it is very sensitive to their proximity. If duplex formation moves the labels even slightly apart energy transfer between them will decline dramatically. The effect is well known to those of skill in the art, and it has been used to measure the relative motion of different parts of various molecules, such as polynucleotides and proteins. Its application in accordance with the present invention can proceed using much the same kinds of techniques, adapted to the solid phase array.

DETECTION

A wide variety of detection techniques may be employed in the invention for reading-out analyte determinations on a chip. The detection techniques, moreover, may rely on any of a wide variety of detectable labels. Such detection techniques and detectable labels are well know and are described in numerous primary publications, technical manuals, laboratory handbooks and textbooks, to name a few. These detection technologies are described in such publications both generally and with respect to specific types of assays and assay procedures. In general, such techniques can be employed in the present invention, particularly those that have already been adapted to detection of binding to genechips and other types of solid-state arrays. Such techniques are described in, for instance, BIOSENSORS, AN INTRODUCTION, Wiley and Teubner, Chichester and Stuttgart (1996), HANDBOOK OF BIOSENSORS AND ELECTRONIC NOSES, MEDICINE, FOOD, AND THE ENVIRONMENT, Erika Kress-Rogers, ed., CRC Press, Boca Raton (1997) and IMMUNOCHEMICAL ASSAYS AND BIOSENSOR TECHNOLOGY FOR THE 1990S, Nakamura et al., eds., American Society for Microbiology, Washington, D.C. (1992), each of which is herein incorporated by reference in its entirety in parts pertinent to detection.

Generally techniques for detection in accordance with the invention can determine both the presence of a detectable label and its position, relative to other positions in an array on a chip. Both abilities can be attained by a variety of means. For instance, detection may involve acquiring an image of a chip that comprises an image of the detectable label in each element and/or assay area, and then, by image processing, determining the results for each assay. Such image processing may be quantitative, threshold-related or qualitative. Alternatively, the chip may be scanned, raster fashion, or quasi-raster fashion, or along a path (ray-tracing mode) so detection is carried out discretely for each element and/or assay area. Each element and/or assay area thus may be detected one or more times in the same or in different portions, and the detection may involve averaging or otherwise processing such repeated samplings. The sampling furthermore may involve detection of each element and/or assay area along a beam line, such as for an absorption assay, or it may involve stimulation by a beam line and detection of an emitted signal, such as fluorescence. In the latter case, the emitted signal may be measured over a spot or a wide area. In addition, the entire chip (or portions thereof) can be excited en banc and the signal coming from elements and/or assay areas then measured individually. Of course, several beams and detectors can be arranged to work in parallel in any such detection paradigm. Spatial resolution may be obtained by tracking the motion and the position of a scanning beam (or other scanning means) as it moves with respect to a chip, by tracking the position of the chip as it moves with respect to the beam, or by a combination of the two. In addition, alignment markers integrated into the chip, sub-assembly, matrix and/or matrix carrier can provide internal alignment and registration signals that can be used by the detector and its associated hardware and software to correlate elements and assays on a chip with the detected signals. Such alignment and registration signals can be incorporated into chips as pseudo-analytes at the time the chip is made. Alternatively or in addition, the markers can be “etched” or written onto the chip outside the elements and the element-containing portion of the assay areas at the time the chips are made. A wide variety of other approaches also may be usefully employed in the invention. Suitability of detection techniques for use in the invention can be assessed by reference to the following parameters.

The detection accuracy should be high. Preferably, the accuracy is within 2-3% deviation or better about a norm for repeated sampling of the same test result.

For clinical applications, and other real-time applications relatively short read-out times are preferred. Thus, for instance, for clinical and other real time applications readout times under an hour, preferably less than 15 minutes and especially preferably less than 5 or I minute are preferred.

To accommodate differing analyte concentrations detection techniques that have a wide dynamic range are preferable.

At best, analyte binding to every assay area on a chip can be detected and/or determined for each sample.

The detector must be able to resolve the elements and assay areas, and it must be able to detect the signal in such areas that samples for analysis will provide.

As noted elsewhere herein, they need not be square but can be any shape. Moreover, also as noted elsewhere herein, size is limited not by intrinsic aspects of the methods and systems herein disclosed but by technical considerations and limitations on fabrication equipment, materials and techniques. Those of skill readily will appreciate and apply to the invention improved materials and techniques as they become available. Such application specifically is contemplated by the present invention.

Many detectors for use in the invention presently are available. Suitable detectors range from inexpensive scanners for use with PC's to sophisticated photon counting imaging microscopes. As noted above, different detectors will be preferred in different embodiments of the invention. Thus, for economical mass production greater economy will be desirable. For other applications very high resolution, sensitivity and dynamic range may be more important. The few examples below, therefore, are merely illustrative.

Sophisticated and sensitive apparatus for detection is available that can be used for clinical and other applications in which large numbers of samples are analyzed, or where equipment expense is not as important as sensitivity, speed and accuracy. Thus, for instance, fluorescence detectors of the type used on automated DNA sequences can be adapted for use in the present invention, for detection of one, two, three, four or even more fluorescent dyes. Likewise, the fluorescence detectors described by Affymax and Affymetrics for detecting binding to peptide and oligonucleotide arrays are readily adaptable for use in accordance with the present invention. Likewise, many laser scanning detectors now used for biochip detection, including laser-ablation mass spectroscopy scanning detectors, are readily employed as detectors in accordance with the present invention.

Another approach might be taken for mass producing detectors, that also illustrates some considerations for implementing both simple and more sophisticated detection regimes and systems. This approach, illustratively discussed below, is to use a scanner to capture the results of assays. A variety of scanners are commercially available, largely for digitizing text and drawings for entry in a computer file associated with document production. Nonetheless, the devices are well suited to capturing any image in digitized, computer readable form.

The devices have in common that they scan an image surface with a laser beam and measure the light reflected from the surface along the scan path. The scan line typically is very thin, 1/100 to 1/400 inches in inexpensive scanners. The full image is digitized by serially scanning the image surface in the direction perpendicular to the scan line. Thus, scanners typically move paper in steps after each line scan to build up the two dimensional image of the paper surface. With hand-held scanners the perpendicular movement is performed manually. It will be appreciated that the scanning mechanism is compact and economical in these devices.

A relatively inexpensive hand-held color scanner can resolve 400 dpi in both dimensions, i.e., 16,000 pixels per square inch. Resolution is in three colors with a grey scale range of 256 levels. Consumer devices are available for digitizing 35 mm color photographs for manipulation on a PC. A typical full color, 12-bit, 2,000 dpi device resolves 4,000,000 12-bit pixels per square inch. More expensive models can achieve very much higher resolution. One device has been reported that can resolve 25,000 dpi, i.e., 625,000,000 pixels per square inch.

EMBEDDED DATA

The detection system can read coded information on the chip in addition to assay test results. Many types of information can be coded, in many formats. One type of information is positional, such as the alignment and registration information discussed elsewhere herein. Such information primarily is useful to allow identification of elements and assay areas with the detected binding, so that signals can be matched to corresponding elements and/or assays. Such information generally can be imprinted at the time the chip is manufactured. Another type of information provides information about the chip itself—its manufacturing lot, for instance. This information, at least in part, can be used to match software to chips so that the results of assays can be calculated and accurately reported. Thus, for instance, the information about each element and assay area in each lot of chips on a given carrier can be recorded in software. The software thus can contain the identity, location, standardization curves and other information about each chip in the carrier necessary for read-out of assay results. The software thus allows great flexibility to chip manufacture, since the location of elements and assays, as well as standardization can be changed from lot to lot in a way that is transparent to the end-user. The ability to identify carriers and chips and match the software to the detected lots provides a way to do this. Such information can include expiration dates for elements, assays and/or carriers that would key an instrument to exclude their use. A third type of “embedded” information can be provided by the user at the time of use, such as a clinician at the time sample is applied. It is highly preferable for the instrument of analysis to be able to capture these types of information, as well as detect the signals arising from analyte binding.

Such information can be embedded in chips, sub-assemblies, matrixes, matrix carriers, and the like in many ways. It can be incorporated during manufacturing of chips by the use of pseudo-analytes. These are signal-generating molecules, compounds, compositions, materials, substances or the like, that generate a position-specific signal for detection. The signal may be intrinsic thereto or it may arise from the analytical process, along with the signals from genuine analytes. Such embedding may be in elements of an assay area or it may be outside assay areas or both. In fact, it can in principle be anywhere on the chip. Specific areas on a chip may be dedicated to these markers, so that the signals always arise from the same areas on chips and/or the markers can be incorporated into different places on different chips or chip runs. These locations can be dedicated to the markers or they can be areas that could also contain ABRs. Moreover, the signal from the markers can be detected in the same way as analyte binding is detected or it can be detected differently. For instance, one or more colors in a multicolor detection scheme can be used exclusively for marker detection. When such a multiplexing approach is used, the markers may even be incorporated with ABRs in elements of a chip. Particularly accurate alignment can be facilitated by multiplexing.

Such information also can be directly imprinted on chips, within a manufacturing ribbon, the individual fibers of a fiber array or in a given coherent fiber bundle. Particularly, certain kinds of lot and registration information may be so imprinted. The information may also be incorporated at the time of dicing or wafering.

Information of this type may take many forms, but preferably is machine detectable and readable by computer systems. Thus, the information can be in the form of shapes, bar codes and/or print, that can be acquired and interpreted by the detector and computer analysis methods and systems useful in the invention. Generally well know techniques useful for these purposes can be employed in this regard in the present invention.

FABRICATING ARRAYS FOR ANALYTE-BINDING ANALYSIS

Fabrication techniques useful in the invention are described in detail elsewhere herein. The following discussion exemplifies application of the aforementioned techniques to fabricating array for analyte binding analysis. The discussion here is shorter than the more detailed discussion above, simply for the sake of brevity. All of the fabrication methods herein disclosed are useful for making array for analyte binding analysis.

In some preferred embodiments for fabricating arrays for analyte binding analysis the analyte binding reagents are comprised in fibers. The fibers are assembled into a coherent bundle, and chips, comprising arrays (also referred to as wafers and unit surfaces) are produced by cross-sectioning the bundle. The fibers define assay areas in the chip. The fiber interiors comprising the assay can be hollow, so that the assay area is on the inner surface of the fiber where it is exposed to the hollow interior (also referred to as the core of the fiber). Alternatively, the interior, or core, of the fiber may be a gel or a matrix or a solid in which the assay areas are embedded and which become exposed surfaces upon cross-sectioning the fiber bundle. Moreover, the fibers may be unitary or may be formed from sub-fibers or may contain additional interior surfaces to, for instance, increase surface area. For instance, fibers may be drawn for micro-channeled material such as “nanochannel” glass. In such embodiments the fibers are themselves bundles of fibers (also referred to as sub-fibers and channels). The fiber itself may be formed from one or more concentric layers, which may have the same appearance on cross-sectioning, or different appearances. The layers may be uniform all around the fiber or may be patterned in a manner that can be detected in cross section. Such patterning can be used to facilitate registration and alignment in assembly and use of the wafers and may be used to facilitate assay identification during read-out of sample analyses. The outside of the fibers may be smooth all around, or it may be patterned to facilitate assembly of the coherent fiber bundle. For instance, the outer surface of the fibers may be formed to present slot and key fittings on different sides that define directionally and/or positionally unique matings with other fibers.

Chips can be produced in accordance with the present invention by assembling fibers comprised or comprising ABRs into a coherent bundle and then making thin cross-sections of the bundle. The cross sections expose the immobilized ABRs in each fiber and provide a surface suitable for sample analysis. The wafers thus produced may provide not only a relatively two dimensional surface for sample analysis but also may provide a surface with depressions or wells, or a flow-through chip.

In a preferred embodiment of this aspect of the present invention hollow fibers are formed. In a particularly preferred embodiment of this aspect of the invention the ABR is introduced into the fiber in the form of a curable gel. The ABR must be stable in the gel. Also the ABR must be homogeneously dispersed in the gel. The gel must have relatively low viscosity to permit flow in the confines of the narrow diameter fiber. The gel must be curable by gentle chemical or physical stimuli. Finally, the gel preferably is optically clear. The fiber is filled with the ABR homogeneously dispersed in the gel, and the gel then is cured.

Many fibers containing different ABRs are fabricated in this way and then assembled into a coherent fiber bundle. The position of each fiber in the bundle is recorded when the bundle is assembled. Later the position of each fiber and the ABR it contains is confirmed by quality control tests, as described below.

After it is assembled, the coherent fiber bundle is sliced across its longitudinal axis, either perpendicularly or obliquely to produce thin wafers. The gels within each hollow fiber in the bundle are exposed on the surface of the wafers. The position of each ABR on the wafer surface is determined by its position in the coherent fiber bundle. Thus, the wafer provides a surface having immobilized in a predetermined pattern thereon the ABRs for simultaneously carrying out multiple analyte determinations on a single sample.

This manufacturing technique has a number of surprising features in terms of reagent use and wafer yield. For instance, a fiber with a hollow core having a diameter of 20 micrometers will have a cross sectional area of about 1.6×10⁻⁴ mm². Since 1,000 mm² is the volume of a milliliter, one mL would fill a length of this fiber of about 6×10⁶ mm. That is to say, 1 mL of a gel containing an ABR would be enough to fill 6 kilometers of the fiber.

If fibers of this geometry are assembled into a coherent fiber bundle and sliced into wafers of 0.2 mm thickness, a single coherent fiber bundle (albeit 6 km long) made from just 1 mL of each ABR would provide 5,000,000 wafers. The economy is independent of the length of the fiber bundle, of course. The yields for larger core diameters are lower but nonetheless less striking. One mL would fill 254 meters (0.245 km) of fiber with a core of 100 micrometers, enough for about 1,275,000 wafers of 0.2 mm thickness. Protein concentrations of about 1 to 5 μg/ml are used in most protocols for coating ABRs such as antibodies onto plastic surfaces. Therefore, in a surface binding procedure using an ABR at 5 μg/ml, 5 μg of the ABR would be enough to fabricate 1, 275,000 wafers 0.2 mm thick. A gel-filling procedure of the type discussed below might use a lower concentration of protein and therefore even less ABR.

An alternative in this approach to gel-filled tubes is surface activated fibers in which an ABR is immobilized on the inner surface of the fiber.

Either during fiber formation or thereafter the inner surface of the fiber is treated so that it will bind an ABR in a uniform and reliable way. And the ABR is immobilized on the inner surface. The uniformity of the ABR coating on the inner surface is carefully measured to insure parameters necessary for proper functioning of the ABR in an assay are fulfilled.

### EXAMPLES

### Example 1

### Oligonucleotide Array 1

8,000 pieces are obtained of silica tubing with OD 250 micrometers, ID 150 micrometers and length 10 meters. The pieces are precisely shaped all along their length. Structural pieces are obtained to facilitate assembly of the tubing into ribbons and bundles. The pieces hold the tubing and, in cross section, provide an asymmetrical pattern in the wafers and arrays produced from the bundle.

8,000 genes of interest are identified. A set of 8,000 oligonucleotide probes consisting of 20 probes and 20 paired single base pair mismatch probes per gene is designed to detect the gene sequences specifically. The probes are obtained in purified form from a commercial supplier of DNA oligonucleotides. Each of the probes is bound separately to a solution of a monomer. Each monomer solution carrying a different probe is introduced into a different tube and polymerized.

The tubes are cut into 1 meter lengths, for convenience, and assembled into bundle as follows. The tubes first are assembled into ribbons using the plastic pieces to facilitate alignment. During assembly, the position of each tube is recorded, as well as the location of the plastic pieces so that the asymmetric pattern can be used to facilitate identifying oligonucleotides in arrays. The ribbons similarly are assembled into bundles. The tubes are aligned side to side, in the same arrangement throughout the bundle. The assembly process is carried out much as pictured in FIGS. 1-4.

The bundle is sectioned perpendicular to the long axis of the tubes (i.e., perpendicular to the alignment axis) at intervals of 100 micrometers to produce wafers somewhat less than 100 micrometers thick (due to loss during cutting). Approximately, 100,000 wafers having the same array of 8,000 probes is produced from all 10 meters of the bundle. The surfaces of the wafers are cleaned and covered with a preserving layer of thin plastic.

### Example 2

### Oligonucleotide Array 2

Arrays are produced in much the same way as described in Example 1; but with the following differences. The probes are not bound to monomers. Instead they are introduced into and bound to the inner surface of the tubes.

### Example 3

### Oligonucleotide Array 3

Arrays are produced in much the same way as described in Example 1; but with the following differences. The probes are not bound to monomers. Instead they are introduced into and bound to the inner surface of the tubes. The tubes then are filled with an support material and assembled into bundles. After the bundles are wafering, each wafer is treated to dissolve the support material.

### Example 4

### Oligonucleotide Array 4

Arrays are produced in much the same way as described in Example 1, with the following differences. The oligonucleotides are synthesized in the tubes from a covalently bound starting nucleotide using standard solid phase oligonucleotide synthesis techniques.

### Example 5

### Oligonucleotide Array 5

Arrays are produced in much the same way as described in Example 4; but with the following differences. After synthesis of the oligonucleotides, the tubes then are filled with an support material and assembled into bundles. After the bundles are wafering, each wafer is treated to dissolve the support material.

### Example 6

### Expression Profiling

Arrays produced as described in Example 1 is mounted in a device of the type shown in FIG. 7. Replicate cell cultures are cultivated and exposed to candidate pharmaceutical agents from a combinatorial library. RNA is prepared from each culture and a labeled hybridization sample is prepared from the total RNA. Sample preparations from cultures exposed to each candidate are hybridized to arrays under stringent conditions. Hybridization of each sample preparation to the array is detected and quantified. Candidates that alter expression of genes in the array are identified.

### Example 7

### Antibody Array

1,000 pieces are obtained of silica tubing with OD 500 micrometers, ID 320 micrometers and length 10 meters. (Scientific Instrument Services). The pieces are precisely shaped all along their length. Structural pieces are obtained to facilitate assembly of the tubing into ribbons and bundles. The pieces hold the tubing and, in cross section, provide an asymmetrical pattern in the wafers and arrays produced from the bundle.

A set of 1,000 antibody preparation of interest is obtained. Each antibody preparation is highly specific for a different human protein. Each of the antibodies is bound separately to a solution of a monomer. Each monomer solution carrying a different antibody preparation is introduced into a different tube and polymerized.

The tubes then are assembled into ribbons using the plastic pieces to facilitate alignment. (To facilitate processing the tubes first are cut into one meter lengths and several bundles are assembled as follows. Each bundle contains one length of tube for each antibody in this example.) During assembly, the position of each tube is recorded, as well as the location of the plastic pieces so that the asymmetric pattern can be used to facilitate identifying the positions of the antibodies in the arrays. The ribbons similarly are assembled into bundles. The tubes are aligned side to side in the bundle in the same arrangement throughout the bundle.

The bundle is sectioned perpendicular to the long axis of the tubes (i.e., perpendicular to the alignment axis) at intervals of 100 micrometers to produce wafers somewhat less than 100 micrometers thick (due to loss during cutting). Approximately, 100,000 wafers having the same array of 1,000 probes is produced from all 10 meters of the bundle. The surfaces of the wafers are cleaned gently and covered with a preserving layer of thin plastic.

### Example 8

### Antibody Array 2

Arrays are produced in much the same way as described in Example 7; but with the following differences. The antibodies are not bound to monomers. Instead they are introduced into and bound to the inner surface of the tubes.

### Example 9

### Antibody Array 3

Arrays are produced in much the same way as described in Example 8; but with the following differences. After antibodies are bound, he tubes then are filled with a support material and assembled into bundles. After the bundles are wafered, each wafer is treated to dissolve the support material.

### Example 10

### Proteomic Profiling

Arrays produced as described in Example 7 is mounted in a device of the type shown in FIG. 7. Replicate cell cultures are cultivated and exposed to candidate pharmaceutical agents from a combinatorial library. Protein sample s are prepared from each culture and labeled. Sample preparations from cultures exposed to each candidate are incubated with arrays under conditions effective for cognate antibodies and proteins to bind one another. The arrays then are washed. Binding of proteins to the arrays are detected and quantified. Candidate agents that alter the protein representation in the cells are identified.

### Example 11

### Clinical Profiling 1

Arrays are produced as described in Example 1, mounted in a device as shown in FIG. 7 and hybridization assays are carried much as described in Example 6, with the following differences. The probes are specific for diagnostic DNA sequences of HIV. The samples are prepared from patients to be tested for HIV markers detectable by hybridization to the array. Hybridization of samples to the array is clinically diagnostic of HIV infection and provides information about the HIV infecting strain and indicates which treatments are most likely to be effective.

### Example 12

### Clinical Profiling 2

Arrays are produced as described in Example 7, mounted in a device as shown in FIG. 7 and protein-binding assays are carried much as described in Example 10, with the following differences. The antibodies are specific for diagnostic markers associated with HIV infection and AIDs. The samples are prepared from patients to be tested for HIV markers and profiled for AIDs markers detectable by the antibodies in the array. Binding of label from samples to the array is clinically diagnostic of HIV infection and provides information about the HIV infecting strain and indicates which treatments are most likely to be effective.

What I claim is:

1. A method for making arrays of a plurality of array members, comprising the steps of:
(A) providing a plurality of array members;
(B) forming bundle members comprising the array members;
(C) assembling the bundle members to form a bundle in which the array members are aligned;
(D) sectioning the bundle to produce wafers that comprise an array of the array members.

2. A method according to claim 1, wherein the array members are cross-sectioned perpendicular to their alignment.

3. A method according to claim 1, wherein the array members are cross-sectioned at an angle of 10 to 80 degrees or 100 to 170 degrees to their alignment.

4. A method according to any of the foregoing claims, wherein the array members are cross-sectioned by a smooth planar cut.

5. A method according to any of claims 1 to 3, wherein the array members are cross-sectioned by a non-planar cut.

6. A method according to claim 5, wherein the surface area of array members exposed by cross-sectioning is increased over that provided by a smooth, planar cut.

7. A method according to claim 1, wherein array members are comprised of or are disposed within a plastic, a glass, a metal or a ceramic.

8. A method according to claim 7, wherein array members are comprised of or disposed within a glass.

9. A method according to claim 7, wherein array members are comprised of or disposed within a plastic.

10. A method according to claim 9, wherein the plastic is a polycarbonate, polyethylene, polymethylmethacrylate, polystyrene, a copolymer of polystyrene, polysulfone, polyvinylchloride, polyester, polyamide, polyacetal, polyethyleneterephthalate, polytetrafluoroethylene or polyurethane.

11. A method according to claim 10, wherein the plastic is a polycarbonate, polyethylene, polystyrene, a copolymer of polystyrene, polysulfone or polyvinylchloride.

12. A method according to claim 1, wherein the array members are spaced about 1.0 to about 1,000 micrometers apart.

13. A method according to claim 1, wherein the array members have a cross-sectional area of about 1.0 to about 1,000,000 μm².

14. A method according to claim 1, wherein the density of array members in the array is about 250 to about 2,500,000 array members per square centimeter of cross sectional surface area of the array.

15. A method according to claim 1, wherein the density in the array is about 10 to about 100,000 array members per square centimeter of total surface area at the assay.

16. A method according to claim 1, wherein there are about 100 to about 2,500,000 aligned array members.

17. A method according to claim 1, wherein there are about 100 to 2,500,000 different aligned array members.

18. A method according to claim 1, wherein cross-sectioning produces sections about 2.5 to about 2,500 micrometers thick.

19. A method for making arrays, comprising the step of cross-sectioning a plurality of aligned array members comprising at least two array members different from one another.

20. A method for making replica arrays, comprising repeatedly cross-sectioning a plurality of aligned array members to produce sections with at least one surface that exposes array members in the same disposition, thereby replicating the array.

21. A method for making arrays for detecting a plurality of analytes, comprising the steps of:
(A) providing a plurality of analyte binding reagents array members;
(B) forming bundle members comprising of or comprising the array members;
(C) assembling the bundle members to form a bundle in which the array members are aligned;
(D) sectioning the bundle to produce wafers that comprise an array of the analyte binding reagents.

22. A method according to claim 21, wherein the array members are cross-sectioned perpendicular to their alignment.

23. A method according to claim 21, wherein the array members are cross-sectioned at an angle of 10-80 degrees or 100-170 degrees to their alignment.

24. A method according to any of claims 21 to 23, wherein the array members are cross-sectioned by a smooth planar cut.

25. A method according to any of claims 21 to 23, wherein the array members are cross-sectioned by a non-planar cut.

26. A method according to claim 25, wherein the surface area of array members exposed by cross-sectioning is increased over that provided by a smooth, planar cut.

27. A method according to claim 21, wherein array members are comprised of or are disposed within a plastic, a glass, a metal or a ceramic.

28. A method according to claim 27, wherein array members are comprised of or disposed within a glass.

29. A method according to claim 27, wherein array members are comprised of or disposed within a plastic.

30. A method according to claim 29, wherein the plastic is a polycarbonate, polyethylene, polymethylmethacrylate, polystyrene, a copolymer of polystyrene, polysulfone, polyvinylchloride, polyester, polyamide, polyacetal, polyethyleneterephthalate, polytetrafluoroethylene or polyurethane.

31. A method according to claim 30, wherein the plastic is a polycarbonate, polyethylene, polystyrene, a copolymer of polystyrene, polysulfone or polyvinylchloride.

32. A method according to claim 21, wherein the array members are spaced about 1.0 to about 1,000 micrometers apart.

33. A method according to claim 21, wherein the array members have a cross-sectional area of about 1.0 to about 1,000,000 μm².

34. A method according to claim 21, wherein the density of array members in the array is about 250 to about 2,500,000 array members per square centimeter of cross sectional surface area of the assay.

35. A method according to claim 21, wherein the density in the array is about 10 to about 100,000 array members per square centimeter of total surface area of the array.

36. A method according to claim 21, wherein there are about 100 to about 2,500,000 aligned array members in the plurality.

37. A method according to claim 21, wherein there are 100 to about 2,500,000 different aligned array members in the plurality.

38. A method according to claim 21, wherein cross-sectioning produces sections about 2.5 to about 2,500,000 micrometers thick.

39. A method for making replica arrays, comprising repeatedly cross-sectioning a plurality of aligned array members to produce sections with at least one surface that exposes array members in the same disposition, thereby replicating the array.

40. A method for making replica arrays for detecting a plurality of analytes, comprising repeatedly cross-sectioning a plurality of aligned analyte binding reagent array members to produce sections with at least one surface that exposes array members in the same disposition, thereby replicating the array.

41. A method according to claim 21, wherein the array comprises analyte binding reagents that hybridize to DNA or RNA having specific nucleotide sequences.

42. A method according to claim 41, wherein the sequence specific binding reagents are polynucleotides, peptide-nucleic acids or polyamides.

43. A method according to claim 42, wherein the sequence specific binding reagents are oligonucleotides.

43. A method according to claim 21, wherein the array comprises analyte binding reagents that bind specific polypeptides.

44. A method according to claim 43, wherein the polypeptide-specific binding reagents are polyclonal antibodies, monoclonal antibodies, a single chain antibody, or an antigen-binding fragment of an antibody.

45. A method according to claim 21, wherein analyte binding reagents are one or more of a nucleic acid, a polynucleotide, a DNA, an RNA, an oligonucleotide, a protein-nucleic acid, an aptamer, a ribozyme, a nucleic acid-binding polyamide, a protein, a peptide, a polypeptide, a glycoprotein, an antibody, an antibody-derived polypeptide, a receptor protein, a fusion protein, a mutein, a lipid, a polysaccharide, a lectin, a ligand, an antigen or a hapten.

46. A method according to claim 21, wherein the array is used to carry out an immunoassay, a hybridization assay, a ligand-binding assay or receptor-binding assay, or a substrate analog affinity assay.

47. A method according to claim 21, wherein binding to the analyte binding reagents is detected using radioactivity, fluorescence, phosphorescence or chemiluminescence.
